(19) United States
(12) Patent Application Publication
(10) Pub. No.: US 2016/0193386 A1
(43) Pub. Date: Jul. 7, 2016

(54) NEOCARTILAGE CONSTRUCTS USING UNIVERSAL CELLS

(71) Applicant: Histogenics Corporation, Waltham, MA (US)

(72) Inventor: Stephen Richard Kennedy, Andover, MA (US)

(21) Appl. No.: 14/988,004

(22) Filed: Jan. 5, 2016

Related U.S. Application Data
(60) Provisional application No. 62/099,784, filed on Jan. 5, 2015.

(51) Int. Cl. A61L 27/38; C12N 5/077; A61L 27/54

(52) U.S. Cl. CPC A61L 27/3817; A61L 27/54; A61L 27/3895; A61L 27/3852; C12N 5/0655; A61L 2430/06; C12N 2506/1346; C12N 2533/54; C12N 2501/10

(57) ABSTRACT

The invention relates to implantable systems for repairing and restoring cartilage. The invention provides methods and products for cartilage repair that use universal chondrocytes. A universal cell line includes cells such as universal chondrocytes that are not immunogenic or allergenic and can be grown in products suitable for use in a number of different people. Use of the universal chondrocytes allows for new processes and products. Where prior art autologous neocartilage constructs required many small reactors (e.g., at least one culture dish per patient to grow one 34 mm disc per patient), using a universal cell line allows, for example, one large batch of cartilage or neocartilage to be made under uniform conditions.

### CROSS-REFERENCE TO RELATED APPLICATION

This application claims priority to, and the benefit of, U.S. Provisional Patent Application No. 62/099,784, filed, Jan. 5, 2015, the contents of which are incorporated by reference.

### FIELD OF INVENTION

The invention relates to implantable systems for repairing and restoring cartilage.

### BACKGROUND

Traumatic injury to cartilage is common in both active people and the elderly and can be the cause of considerable pain and disability. Existing approaches to treatment include rest and surgical procedures such as micro-fracture, drilling, and abrasion. Unfortunately, such approaches typically only provide temporary relief to symptoms. Severe cases of cartilage injury may require joint replacement. An estimated 200,000 knee-replacements are done each year. An artificial joint typically lasts less than 15 years and so is usually not recommended for people under fifty. Some treatment approaches seek to use synthetic cartilage

U.S. Pat. No. 5,723,331 describes making synthetic cartilage for cartilage repair by using chondrocytes ex vivo. Those cells are meant to secrete cartilage-specific extracellular matrix but that extracellular matrix may be found lacking in quality. U.S. Pat. No. 5,786,217 reports a multi-cell cartilage patch made ex vivo with non-differentiated cells which are then cultured to allow the cells to differentiate. U.S. Pub. 2002/0082220 reports on repairing cartilage by introducing into tissue a temperature dependent polymer gel and a blood component to promote cell proliferation. U.S. Pat. No. 6,528,052 reports generating cartilage by trying to mimic natural loading. Unfortunately, none of the prior art methods result in a product of optimal quality.

U.S. Pat. No. 8,906,686 reports a neocartilage construct made by culturing donor chondrocytes in conditions that benefit the quality of the extracellular matrix. Using donor chondrocytes creates an autologous product in that a patient is treated with a construct made from their own cells.

Unfortunately, using patients' cells to grow autologous constructs present significant problems. Not only do cells from some patients not grow well (and sometimes fail to grow at all), there is much variability in what different cells from different individuals will need. Thus where a biomaterial is prepared for a large number of individuals, for example tens of thousands of patients per year, collecting and using donor cells from each of those people presents significant problems in terms of culturing those cells with existing techniques.

### SUMMARY

The invention provides methods and products for cartilage repair that use universal chondrocytes differentiated from stem cells. A universal cell line includes cells such as universal chondrocytes that are not immunogenic or allergenic and can be grown in products suitable for use in a number of different people. Use of the universal chondrocytes allows for new processes and products. Where prior art autologous neocartilage constructs required many small reactors (e.g., at least one culture dish per patient to grow one 34 mm disc per patient), using a universal cell line allows, for example, one large batch of cartilage or neocartilage to be made under uniform conditions. Production conditions can include suspending cells from the universal cell line in a collagen solution and making a coating or casting of collagen or collagen gel by incubating in appropriate conditions for temperature, pressure, pH, salinity, co-factors, etc.

Aspects of the invention provide methods of making a volume of neocartilage in, for example, the form of a sheet. For sheets of cartilage, collagen gel or solution and chondrocytes are incubated in a reactor to form the sheet. The sheet is harvested and cut into pieces to be used as inserts for cartilage repair. A sheet of cartilage may include additional materials such as nanoparticles such as liposomes which may themselves include other materials such as nutrients, growth factors, antibodies, drugs, steroids, anti-inflammatories, etc., to provide a controlled release mechanism for inserts cut from the sheet. Example treatment uses for neocartilage inserts cut from a sheet may include osteoarthritis treatment or hip, spine, knee, etc., treatment. Where materials of the invention are being used to treat Rheumatoid Arthritis, for example, antibodies or steroids may be included to control an autoimmune response and stop progression of the condition. In some embodiments, sheets of cartilage or neocartilage are prepared by universal cells in a monolayer sheet (2D culture) in the presence of a bioactive agent under conditions sufficient for inducing proliferation and differentiation of the cell sample. After 2D culture, at least a portion of the proliferated and differentiated cells can be isolated from the monolayer culture and suspended. The cells in the suspension may also be referred to as a suspension matrix. The suspended cells may then seeded into a scaffold. The seeded scaffold may then be subject to culturing conditions sufficient for inducing maturation of the cells into cartilage. In certain embodiments, the culturing conditions are static and exclude the application of a mechanical stimulus. In other embodiments, the culturing conditions include the application of a mechanical stimulus, such as hydrostatic pressure. Methods may provide a neocartilage construct suitable for implantation into a cartilage lesion in situ, e.g., under one or between two layers of biologically acceptable sealants within a cartilage lesion.

In certain aspects, the invention provides a method of making implants for cartilage repair. The method includes introducing a composition comprising collagen and a plurality of living universal chondrocytes into a tissue reactor; incubating the composition to form a bulk implant material; excising a first implant from the bulk implant material, wherein the first implant comprises a first portion of the living universal chondrocytes and is suitable for implantation into a first human patient; and excising a second implant from the bulk implant material, wherein the second implant comprises a second portion of the living universal chondrocytes and is suitable for implantation into a second human patient. The method may include differentiating pluripotent stem cells into the living universal chondrocytes prior to the introducing step (or during, after, overlapping with, or a combination thereof). The plurality of living universal chondrocytes thus may be differentiated pluripotent stem cells. In preferred embodiments, the composition includes a porous primary scaffold made with the collagen and having a plurality of pores, and the introducing step further includes introducing a solution comprising a second collagen and the plurality of living universal chondrocytes into the plurality of pores. Incubating the composition stabilizes the solution to form a fibrous, cross-linked network comprising the second collagen within the plurality of pores. In some embodiments, the solution comprises a basic pH and a surfactant.

The bulk implant material may be provided as a 2D sheet, i.e., a sheet less than 5 mm thick and greater than tens of cm by tens of cm in area. Methods of the invention may include harvesting at least four different implants for at least four different human patients from the sheet. The sheet may include a plurality of nanoparticles (e.g., nutrients, growth factors, antibodies, drugs, steroids, or anti-inflammatories). The sheet may be prepared using the universal cells in a monolayer, 2D culture in the presence of a bioactive agent under conditions sufficient for inducing proliferation and differentiation of the pluripotent stem cells into the universal chondrocytes.

In preferred embodiments, the collagen and the second collagen each comprise Type I collagen. The solution may further include a bone inducing agent selected from the group consisting of a fibroblast growth factor (FGF), a bone morphogenic protein (BMP), insulin growth factor (IGF), and transforming growth factor beta (TGF-B). Preferably, the porous primary scaffold has a substantially homogeneous defined porosity and wherein each of the plurality of pores have a diameter of about 300±100 μm at an upper surface and a lower surface of the sheet.

Aspects of the invention provide a composition for cartilage repair. The composition includes a bulk implant material that has a porous primary scaffold comprising collagen and a plurality of pores as well as a secondary scaffold comprising a second collagen disposed within the plurality of pores. The composition further includes a plurality of living cells from a universal cell line disposed within the bulk implant material. The bulk implant material is configured such that a plurality of different cartilage repair implants for a plurality of different human patients may be excised from the bulk implant material. Preferably, the bulk implant material is configured such that each of the plurality of different cartilage repair implants may be at least as large as a disc with a diameter of 30 mm and a thickness of 2 mm. In some embodiments, the implants have a thickness of about 2 mm and an area of at least 2 cm̂2. The plurality of living cells may be chondrocytes differentiated from pluripotent stem cells. The porous primary scaffold may have a substantially homogeneous defined porosity and wherein each of the plurality of pores have a diameter of about 300±100 μm at an upper surface and a lower surface of the sheet. The secondary scaffold may have a basic pH and a surfactant. In some embodiments, the collagen and the second collagen each comprise Type I collagen.

In some embodiments, the bulk implant material comprises a sheet less than 5 mm thick and greater than tens of cm by tens of cm in area. The sheet may include a plurality of nanoparticles such as one or more of nutrients, growth factors, antibodies, drugs, steroids, and anti-inflammatories. Preferably, the sheet is prepared using the plurality of living cells in a monolayer, 2D culture in the presence of a bioactive agent under conditions sufficient for inducing proliferation and differentiation of the pluripotent stem cells into the chondrocytes.

The secondary scaffold may include a bone inducing agent selected from the group consisting of a fibroblast growth factor (FGF), a bone morphogenic protein (BMP), insulin growth factor (IGF), and transforming growth factor beta (TGF-B). The composition may include one more of a fibroblast growth factor (FGF), a bone morphogenic protein (BMP), insulin growth factor (IGF), and transforming growth factor beta (TGF-B).

In certain embodiments, the plurality of living cells comprises pluripotent stem cells and chondrocytes differentiated from pluripotent stem cells. For example, the plurality of living cells may include pluripotent stem cells actively differentiating into chondrocytes.

In some aspects, the invention provides a kit for the production of neocartilage on-demand. The kit includes: a collagen solution; a porous matrix comprising collagen; and a plurality of living universal cells, all provided to be mixed into a mixture at a treatment location for use in a patient. Within the kit, the plurality of living universal cells may be provided in and as part of the collagen solution. Preferably, the collagen solution comprises one more of a fibroblast growth factor (FGF), a bone morphogenic protein (BMP), insulin growth factor (IGF), and transforming growth factor beta (TGF-B1). The plurality of living universal cells may include pluripotent stem cells and chondrocytes differentiated from pluripotent stem cells and may even include pluripotent stem cells actively differentiating into chondrocytes.

The kit may include a dispenser for delivering the mixture to the patient. The dispenser may be a hand-held device with a handle and a delivery nozzle. The dispenser may be configured to deliver the mixture arthroscopically. Preferably, the collagen is Type I collagen and the collagen solution also comprises Type I collagen. The porous matrix may have a plurality of pores oriented substantially parallel to each other having diameters of about 300±100 μm. The collagen solution may have a basic pH, a surfactant, and one or more chondrogenic growth factors.

Aspects of the invention provide a method of preparing a composition to for use in treating a cartilage defect in a patient, the method comprising using any kit described above to create a mixture to be delivered to and incubated in the cartilage defect in the body of the patient.

In certain aspects, the invention provides a method of creating a neocartilage treatment insert. The method includes obtaining a mixture comprising a collagen solution and cells from a universal cell line and forming an insert from the mixture using a 3D forming device such as a 3D printer or an injection mold. The method may further include taking a 3D image of an affected site by a 3D imaging modality; building a 3D model of the affected site; and forming the insert for the affected site using the 3D model. Preferably, the 3D imaging modality comprises one selected from the group consisting of computed-tomography and ultrasound. The affected site may be one selected from the group consisting of hip, knee, nose, ear, and spinal disc.

Other aspects of the invention provide a reactor for creating a volume of neocartilage. The reactor includes an incubation chamber dimensioned to hold a sheet or mass of material including universal chondrocytes that, once formed, can be portioned into numerous (e.g., dozens or hundreds) of neocartilage inserts. Since the sheet or mass of material is held in a controlled incubation chamber, conditions can be controlled to provide a high-quality product such as neocartilage with a high-quality extracellular matrix while also obviating the need for individually culturing donor cells from numerous different patients in a plurality of different individual reaction chambers. The reactor can include mechanisms to suffuse the sheet or mass of material in nutrient media under a controlled atmosphere. The reactor may be used to control an ionic character of the nascent neocartilage, molecular weight, presence of co-factors or growth factors, treatments such as small molecules, nano-particles, etc.

Aspects of the invention provide neocartilage “on-demand” by using a universal cell line in a product or kit that includes a collagen solution and a matrix that are both supplied (e.g., as a kit) to a clinic to be mixed and used on-site. Such a product or kit provides “on demand” neocartilage which allows for a variety of use and delivery options. The solution may include a collagen solution and the universal cells. Neocartilage on demand may be characterized by having components that are mixed at locations other than a source. Providing the components separately allows the neocartilage components to be provided and then mixed on-site.

In body-as-bioreactor embodiments, neocartilage is mixed and incubated within the patient, in the affected site. Using the patient as incubator may have benefits such as a decreased chance of problems arising from introducing a fully incubated neocartilage insert into a patient. Incubation within a patient allows for different approaches to treating defects. A surgeon may excise damaged cartilage and fill the site with a neocartilage mixture which then incubates in situ. In some embodiments, the invention provides tools for localized delivery of the neocartilage mixture. A dispense, such as a hand-held pressure-based volumetric dispenser can be used. Additionally or alternatively, a mixture may be delivered arthroscopically or laparoscopically. Additionally it is noted that localized delivery of a neocartilage mixture need not require any excision or removal of material for certain repairs or in certain contexts. For example, in some contexts for a small defect, a surgeon may not need to debrided down to the bone but may simply instead go in to the site and treat with a filler from the mixture.

Other aspects of the invention may include creating or using a preserved sample of neocartilage. For example, neocartilage or one of the components of a kit for on-demand neocartilage could be frozen and later re-activated, lyophilized, or otherwise preserved. An additive can be used to preserve molecular integrity and structure.

In some aspects, 3D bio-printing makes use of a universal cell line that is used, maintained, or provided separately from a collagen solution, which allows those materials to be created, stored, distributed, and used for novel methods and products. For example, with non-autologous materials, since donor cells do not need to be individually cultured to create neocartilage insert, the cell line and the solution can be used in a process that includes three-dimensional (3D) printing or similar sculpting fabrication techniques (e.g., injection molding) to make an insert that is customized to a target site. Using methods of 3D bioprinting with methods of the invention for optimizing conditions for developing chondrocytes, customized neocartilage inserts can be made that have a high-quality such as for example having a high-quality extracellular matrix. Methods can include taking a 3D image of an affected site by a 3D imaging modality such as computed-tomography or ultrasound, building a 3D model of the affected site, and creating a customized neocartilage insert for the affected site using the 3D model. Modeling methods may be applicable in the context of a damaged hip, knee, nose, or ear, and may have particular applicability in the context of a spinal disc. Methods of the invention can be used in any context that requires a customized piece of cartilage.

Aspects of the invention may be used with repair scaffold products such as the repair scaffold sold under the trademark VERICART by Histogenics Corporation (Waltham, Mass.). The cartilage repair scaffold may be provided along with universal cells. The scaffold may include an off-the-shelf lyophilized, double structured collagen scaffold for use as a suture-less implant. Materials may include an adhesive (e.g., integrated or as part of a kit) to place and secure the implant as well as universal chondrocytes. The implant is strong and secure and can be used in weight-bearing applications quickly to speed the healing process. The implant may be described as a double-structured tissue implant, which may include a collagen-based double-structured tissue implant comprising a primary scaffold and a secondary scaffold in which the secondary scaffold is a qualitatively different structure formed within confines of the primary scaffold. For example, the implant may use a collagen-based primary porous scaffold having vertical open pores suitable for incorporation of a secondary scaffold. The secondary scaffold may be incorporated into the primary scaffold by introducing a basic solution comprising collagen and a non-ionic surfactant into the primary scaffold and subjecting the product to precipitation, lyophilization and dehydrothermal treatment. One or different ones of the implant product may be independently seeded with cells. Implants may optionally include a pharmaceutical agent, a growth modulator, nanoparticles, or combinations thereof. Additionally or alternatively, the secondary scaffold is provided as a standalone product prepared from a neutralized basic solution (e.g., comprising collagen and a surfactant subjected to lyophilization and dehydrothermal treatment). In certain embodiments, an implant includes a primary porous scaffold prepared from a biocompatible collagen material and in which the scaffold has a substantially homogeneous defined porosity and uniformly distributed randomly and non-randomly organized pores of substantially the same size of defined diameter of about 300±100 μm. Methods include introducing a collagen solution comprising at least one non-ionic surfactant (basic solution) into the pores of said primary scaffold. The collagen solution may be stabilized therein by precipitation or gelling, dehydrated, lyophilized and dehydrothermally processed to form a distinctly structurally and functionally different second scaffold within said pores of said primary scaffold. Discussion may be found in U.S. Pat. No. 8,685,107, incorporated by reference. Use of a double-structured collagen scaffold with universal cells may provide methods of treatment that do not required taking a sample from a donor (such as bone marrow aspirate), which avoids one aspect of prior art methods that causes considerable discomfort and inconvenience to patients. The double structured implant with universal chondrocytes may be implanted or glued into the treatment site to repair cartilage.

Aspects of the invention include neocartilage components, kits, or products as a delivery vehicle for other materials. For example, a collagen solution or gel, a scaffold, or both may be used, any one of which include one or more of growth factors, nanoparticles, nutrients, drugs, other materials, or combinations thereof. For example, nanoparticles can include liposomes or other particles known in the art and the liposomes can include growth factors, nutrients, antibiotics, adjuvants, etc. Those particles (e.g., liposomes) can then provide an extended release mechanism for the materials included therein. A neocartilage insert of the invention, or a double-structured implant, may thus include an extended release mechanism. The extended release mechanism may have particular applicability in the context of patient-as-incubator, in which the collagen solution and a scaffold are mixed and introduced to incubate in situ to grow the neocartilage material.

Methods and materials of the invention thus provide for a body-as-bioreactor treatment scheme. Materials are introduced into the defect that may include universal cells, a collagen solution (e.g., not-yet gelled), and one or any combination of GFs, nutrients, etc., either directly or via nanoparticles. The materials provide for the cells to proliferate to create an extracellular matrix. The body-as-bioreactor may have applicability in diverse settings including, for example, knee-replacement for the elderly or sports-injury repair. Upon introduction of the mixture into the defect site, it may be found that the collagen solution gels within about thirty minutes and that a good extracellular matrix and cartilage are formed within a week or two. Additionally, it may be beneficial to provide the mixture both with universal cells directly as well as universal cells within liposomes for controlled release of those cells.

The various described aspects of the invention thus generally relate to materials for repairing cartilage and methods for preparing the same using a universal cell line that provides, for example, universal chondrocytes, which are non-immunogenic, non-allergenic. One or more bioactive agent may be included to increase the activation and proliferation of chondrocytes and increase sulfated glycosaminoglycan production (sGAG). A higher chondrocyte cell count and increased sGAG expression directly correlate with a more developed extracellular matrix, providing end-materials that better mimic natural cartilage, increasing repair successes and integrating into the implantation site without pathogenesis. In various aspects or embodiments, the invention provides systems and methods for marking and using sheets of cartilage, kits and methods for cartilage on-demand, methods for body-as-bioreactor cartilage repair, double-structure cartilage repair scaffold implants, and cartilage repair materials as delivery vehicle all of which aspects and embodiments preferably employ a universal cell line.

A universal cell line may include any suitable cell type and universal describes cells that are not limited to use in a single patient (i.e., not strictly autologous cells from that patient). Cells suitable for use in systems and methods of the invention include allogeneic or syngeneic heterologous cells. The cells may include, for example, bone marrow aspirates, chondrocytes, fibroblasts, fibrochondrocytes, tenocytes, osteoblasts, stem cells, or a combination thereof. Stems cells suitable for use in systems and methods of the invention include adult stem cells, mesenchymal stem cells, peripheral blood stem cells, induced pluripotent stem cells, or any combination thereof.

Materials of the invention may include a culture medium, suspension, scaffold, or component thereof that includes a bioactive agent such as a fibroblast growth factor. Suitable fibroblast growth factors include FGF2, FGF4, FGF9, FGF18 or variants thereof. Fibroblast growth factors may be included to increase the proliferation of the extracellular matrix components.

Materials of the invention according to some of the above-described aspects and embodiments use a scaffold for supporting proliferation of the universal cells and differentiation of those cells into neocartilage. Scaffolds are also referred to herein as support matrices. Preferably, the scaffold is acellular. In certain embodiments, an acellular, collagen scaffold is a biodegradable collagenous sponge, a honeycomb or honeycomb-like sponge, a thermo-reversible gelation hydrogel. In certain embodiments, a solution, such as collagenous solution, is disposed within the pores of the scaffold. The solution is then stabilized within the pores of the scaffold to create a fibrous collagen network within the scaffold. The scaffold with the fibrous collagen network may be used directly as an implant. Alternatively, a cell suspension may be introduced into the scaffold with the fibrous collagen network and cultured ex-vivo to generate neocartilage.

### BRIEF DESCRIPTION OF THE DRAWINGS

FIG. 1 shows a neocartilage support matrix of collagen embedded with chondrocytes.

FIG. 2 shows a microphotograph of a neocartilage construct.

FIG. 3 shows a rehydrated double-structured tissue implant.

FIG. 4 shows a dry form of the double-structured tissue implant.

FIG. 5 gives a diagram of a tissue processor system.

FIG. 6 shows a tissue engineering support system.

FIG. 7 is a graph illustrating S-GAG production per seeded matrix.

FIG. 8 is a photomicrograph of Safranin-O staining for S-GAG, when the cell constructs are subjected to static atmospheric pressure.

FIG. 9 is a photomicrograph of Safranin-O staining for S-GAG, with cell constructs subjected to a cyclic hydrostatic pressure for 6 days, followed by 12 days of static atmospheric pressure.

FIG. 10 shows the sulfated glycosaminoglycan production in μg/cell construct.

FIG. 11 shows increased production of DNA in constructs processed under cyclic or constant hydrostatic pressure.

FIG. 12 is a graph comparing effect of constant atmospheric pressure (Control) and zero MPa hydrostatic pressure.

FIG. 13 shows the index of DNA content (Initial=1) in matrices subjected to static (Control), zero hydrostatic (0 MPa), cyclic (Cy-HP) or constant (Constant-HP) hydrostatic pressure for 6 day and 12 days of atmospheric pressure culture.

FIG. 14 shows accumulation of S-GAG on day 18 in matrices subjected to atmospheric pressure.

FIG. 15 shows accumulation of S-GAG in matrices subjected to 6 days of cyclic hydrostatic pressure (Cy-HP), followed by 12 days of atmospheric pressure.

FIG. 16 shows accumulation of Type II collagen in matrices subjected to the atmospheric pressure.

FIG. 17 shows accumulation of Type II collagen in matrices subjected to cyclic hydrostatic pressure.

FIG. 18 describes results of studies of the effect of the perfusion flow rate on cell proliferation measured by levels of DNA content index at day 0, 6 and 18.

FIG. 19 describes results of studies of the effect of the perfusion flow rate on cell proliferation measured by S-GAG accumulation at day 0, 6 and 18.

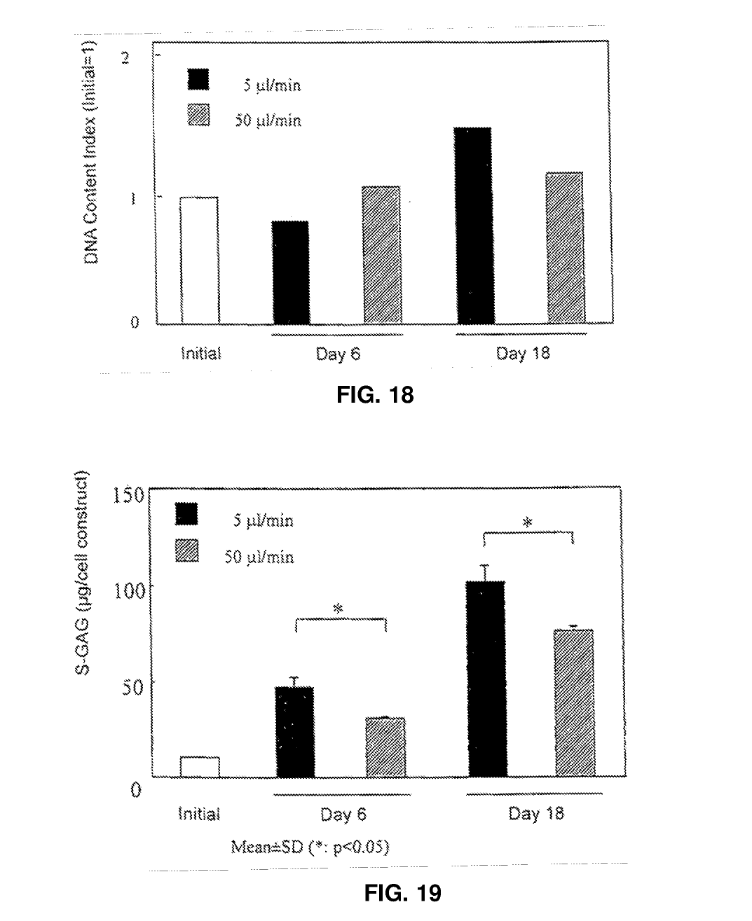

FIG. 18 shows that the lower perfusion rate results in higher DNA content.

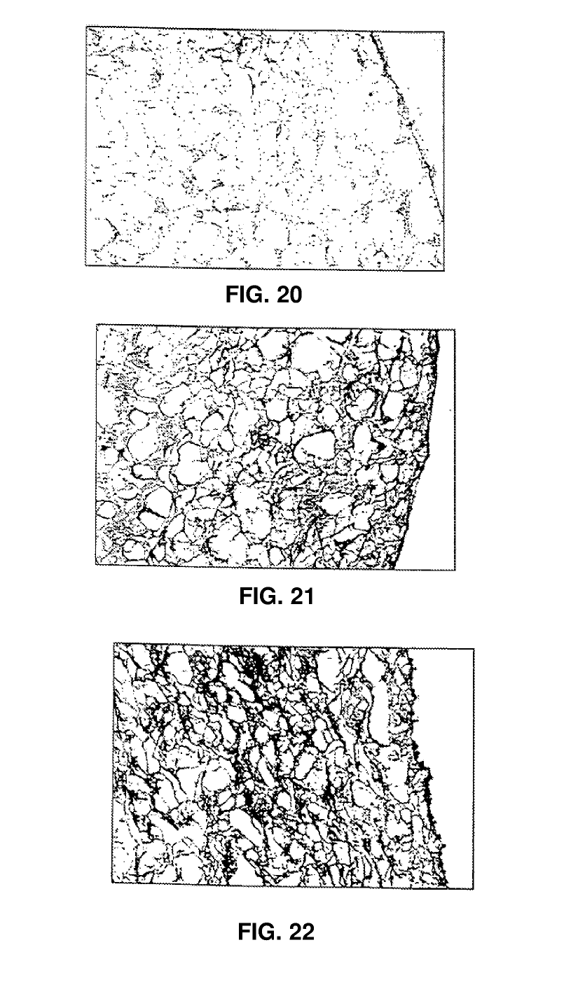

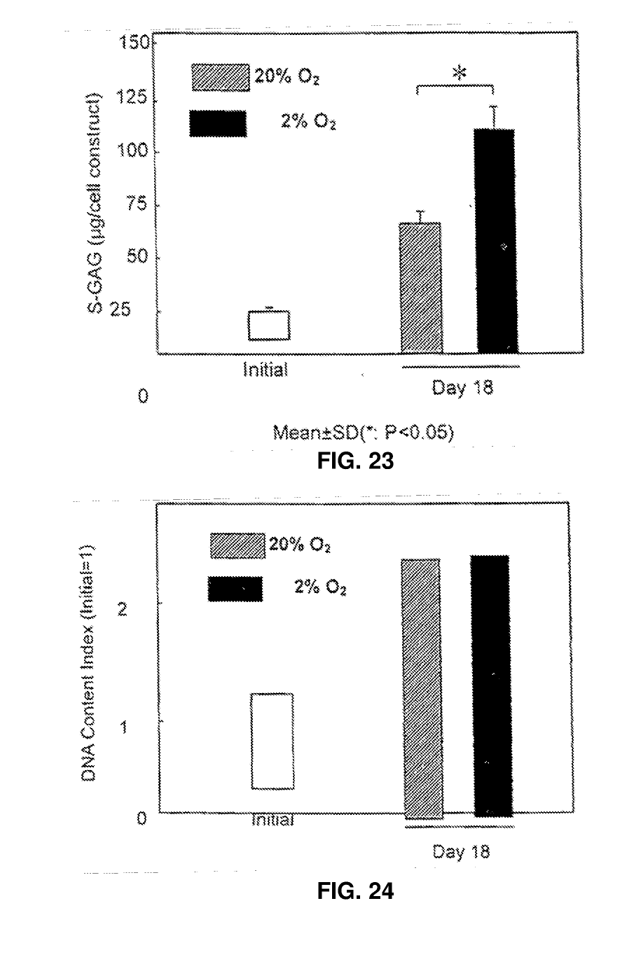

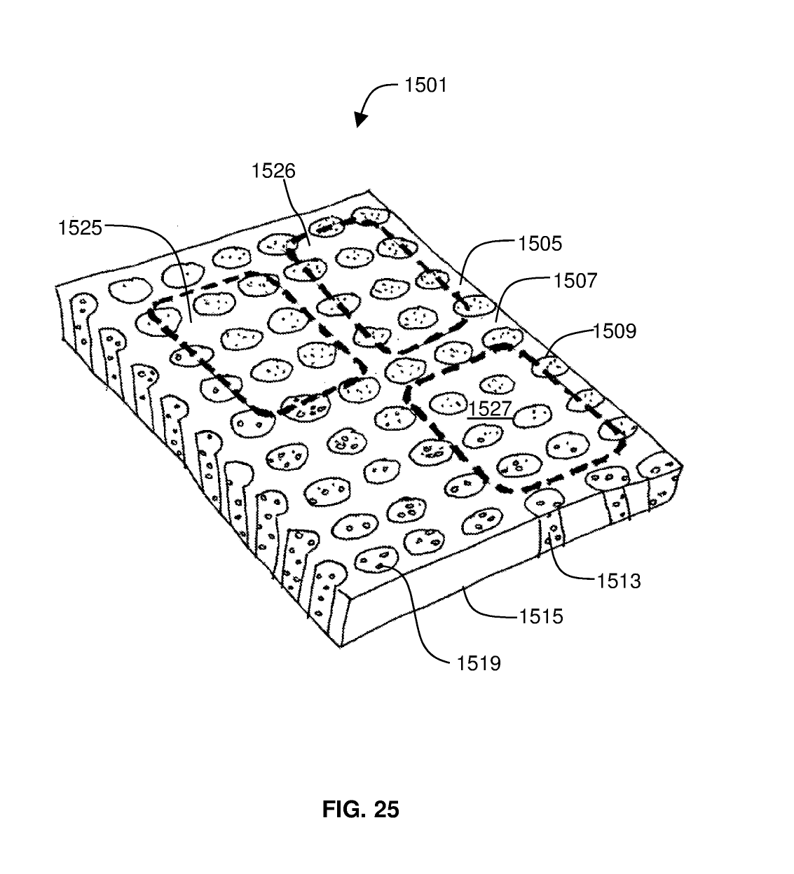

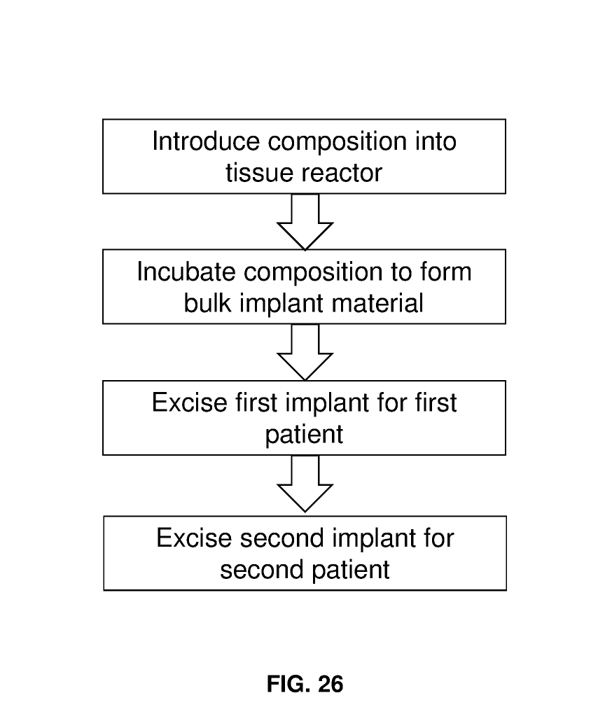

FIG. 19 shows S-GAG for 5 and for 50 μl/min perfusion.

FIG. 20 illustrates a formation of extracellular matrix after 15 days culture determined in matrices treated with perfusion only.

FIG. 21 illustrates a formation of extracellular matrix after 15 days culture determined in matrices treated cyclic hydrostatic pressure 2.8 MPa at 0.015 Hz.

FIG. 22 illustrates a formation of extracellular matrix after 15 days culture.

FIG. 23 is a graph showing S-GAG production by cell constructs subjected to 2% oxygen concentration (Cy-HP) and to cyclic hydrostatic pressure followed by static pressure.

FIG. 24 shows the DNA content index (initial=1) in cell constructs subjected to 2% or 20% oxygen concentration and Cy-HP pressure followed by static pressure.

FIG. 25 depicts a composition for cartilage repair.

FIG. 26 diagrams a method of making implants for cartilage repair.

### DETAILED DESCRIPTION

This invention is based on methods and materials that use a universal cell line such as universal chondrocytes. Universal chondrocytes may be included in neocartilage and upon incorporating this neocartilage into the support matrix and submitting said neocartilage/support matrix to culture methods described herein, the neocartilage/support matrix become a structural unit called neocartilage construct. Such processed neocartilage with universal cells is suitable for implantation into a lesion of injured, traumatized, aged or diseased cartilage optionally under or within sealant layers. Sealant promotes in situ formation of de novo superficial cartilage layer over the cartilage lesion. Use of universal chondrocytes allows neocartilage to be made en masse. For example, sheets of cartilage may be made in a reactor/incubator of the invention. Use of universal cells also allows for solution/cell kits to be created for use at clinics and treatment sites.

The invention thus, in its broadest scope, concerns a method for preparation of neocartilage using universal chondrocytes. Various embodiments of the invention provide methods for formation of a support matrix, methods for fabrication of a neocartilage construct, methods for de novo formation of a superficial cartilage layer in situ, methods for repair and restoration of damaged, injured, traumatized or aged cartilage to its full functionality, and methods for treatment of injuries or diseases caused by damaged cartilage due to the trauma, injury, disease or age.

Briefly, the invention comprises preparation of neocartilage from universal or heterologous chondrocytes, culturing and expansion of chondrocytes, seeding the chondrocytes within a collagenous or thermo-reversible gel support matrix and propagating said chondrocytes in two or three-dimensions. To achieve the chondrocyte propagation, the seeded support matrix is optionally subjected to the algorithm of variable conditions, such as static conditions, constant or cyclic hydrostatic pressure, temperature changes, oxygen and/or carbon dioxide level changes and changes in perfusion flow rate of the culture medium in the presence of various supplements, such as, growth factors, ascorbic acid, ITS, etc. The chondrocyte-seeded support matrix treated as above becomes a neocartilage construct (neocartilage) suitable for implanting into a joint cartilage lesion. Additionally or alternatively, the invention provides materials that can be mixed and delivered at the time of treatment such that the neocartilage forms in situ within the patient under a patient-as-bioreactor strategy.

In some embodiments, a neocartilage construct is implanted into the lesion under a top sealant, or into a cavity formed by two layers of adhesive sealants. The first layer of the sealant is deposited at and covers the bottom of the lesion and its function is to protect the integrity of said lesion from cell migration and from effects of various blood and tissue metabolites and also to form a bottom of the cavity into which the neocartilage construct is deposited.

In one embodiment, after the neocartilage construct is emplaced into the lesion cavity, the second adhesive layer is deposited on the top of the neocartilage construct and within several months results in formation of the superficial cartilage layer completely sealing the lesion.

In the alternative embodiment, two adhesive layers may be deposited concurrently with or before the construct is implanted into the cavity between them. In such an instance, in the interim, said cavity may be filed with a space holding thermo-reversible gel (SHTG). Both sealant layers and the construct or space holding gel are left within the lesion cavity for a certain predetermined period of time, typically from one week to several months, or in case of the space holding gel, until the neocartilage construct is prepared ex vivo and ready to be implanted. The second layer deposited on the top and over the lesion promotes formation of a superficial cartilage layer which covers the lesion on the outside and eventually overgrows the lesion completely thereby resulting in complete or almost complete sealing of the lesion and of the neocartilage construct deposited within said lesion leading to incorporation of neocartilage into a native cartilage and resulting in healing of the injured or damaged cartilage. In alternative, the thermo-reversible gel may serve as an initiator for promotion of formation of the superficial cartilage layer.

Both the support matrix of the neocartilage construct or the space holding thermo-reversible gel deposited into the lesion are materials which are biodegradable and permit and promote formation of the superficial cartilage layer and integration of the chondrocytes from the neocartilage construct into the native cartilage within the lesion cavity. Such integration begins within several weeks or months following the implanting and may continue for several months and involves a growth and maturing of neocartilage into normal cartilage integrated into the healthy cartilage. The top sealant layer promotes an overgrowth of the lesion with the superficial cartilage layer typically in about two-three months when the sealant is itself degraded.

In the alternative embodiment, the lesion cavity is filled with a space-holding gel until the outer superficial cartilage layer is formed at which time the neocartilage construct comprising ex vivo propagated chondrocytes suspended in a thermo-reversible sol is introduced at a temperature between 5° and 15° C. After it is introduced into the lesion as a liquid sol, the introduced thermo-reversible sol-gel is converted into a solid gel at body temperatures of 37° C. or at the same or similar temperature as the temperature of the synovial cavity. The neocartilage construct introduced into the lesion is integrated into the native cartilage surrounding the cavity and is completely covered with the superficial cartilage layer.

In the alternative, the neocartilage construct is deposited into a lesion of injured, traumatized, aged or diseased cartilage over the first (bottom) sealant layer and the thermo-reversible gel of the neocartilage construct promotes in situ formation of the superficial membrane without a need to add the second sealant.

The method for treatment of injured, traumatized, diseased or aged cartilage comprises treating the injured, traumatized, diseased or aged cartilage with an implanted neocartilage construct prepared by methods described above and/or by any combination of steps or components as described.

### I. Preparation of Neocartilage Constructs

Preparation of neocartilage constructs for implanting into the cartilage lesion involves culturing universal chondrocytes, seeding them in the support matrix and preparation thereof, and propagating the chondrocytes either ex vivo, in vitro, or in vivo.

According to certain embodiments, preparation of neocartilage constructs involves introducing bioactive agent into a culture medium, suspension, scaffold, solution disposed within the scaffold, or combinations thereof. For combinations, one or more bioactive agents may be introduced into any one or more of the culture medium, suspension, scaffold, or solution disposed within the scaffold used in methods of the invention without limitation. For example, when cells are cultured in a culture medium while in the presence of a bioactive agent, another bioactive agent may not be introduced into the suspension, scaffold, or solution disposed within the scaffold. Alternatively, when cells are cultured in a culture medium while in the presence of a bioactive agent, the suspension, scaffold, or solution disposed within the scaffold may likewise include a bioactive agent. The one or more bioactive agents disposed in the culture medium, suspension, scaffold, or solution disposed within the scaffold may be the same or different.

The bioactive agent may include a growth factor, a cytokine, a peptide, a matrix remodeling enzyme, a matrix metalloproteinase, an aggrecanase, a cathepsin, demineralized bone powder, calcium phosphate, hydroxyapatite, organoapatite, titanium oxide, poly-L-lactic acid or a copolymer thereof, polyglycolic acid or a copolymer thereof, and a combination thereof. The growth factor may include a fibroblast growth factor (FGF), a bone morphogenetic protein (BMP), insulin growth factor (IGF), transforming growth factor beta (TGF-B), or a combination thereof. In certain embodiments, the bioactive agent is a bone inducing agent (e.g. fibroblast growth factor). Suitable fibroblast growth factors include, for example, FGF2, FGF4, FGF9, FGF18, or variants thereof (e.g. FGF2v1). In addition, suitable growth factors include, for example, growth factors discussed in U.S. Pat. No. 7,288,406; U.S. Pat. No. 7,563,769; U.S. Pub. 2011/0053841; and U.S. Pub 2010/0274362, each incorporated by reference. In particular aspects, a growth factor used in embodiments of the invention is an FGF2 variant. In one embodiment, the FGF2 variant used has the asparagine at position 111 replaced by glycine, and the alanine at position 3 and the serine at position 5 replaced by glutamine, and is denoted as FGF2(3,5Q)-N111G. The amino acid sequence of FGF2v1 is described in U.S. Pub. 2014/0193468. Methods for preparing neocartilage constructs are discussed in more detail hereinafter.

### A. Cartilage and Neocartilage

Cartilage is a connective tissue covering joints and bones. Neocartilage is immature cartilage which eventually, upon deposition into the lesion according to this invention, is integrated into and acquires properties of mature cartilage. Differences between the two types of cartilage lie in their maturity. Cartilage is a mature tissue comprising metabolically active but non-dividing chondrocytes; neocartilage is an immature cartilage comprising metabolically and genetically activated chondrocytes which are able to divide and multiply. This invention utilizes properties of neocartilage in achieving repair and restoration of damaged cartilage into the full functionality of the healthy cartilage by enabling the neocartilage to be integrated into the mature cartilage surrounding the lesion and in this way repair the defect.

i. Cartilage

Cartilage is a connective tissue characterized by its poor vascularity and a firm consistency. Cartilage consists of mature non-dividing chondrocytes (cells), collagen (interstitial matrix of fibers) and a ground proteoglycan substance (glycoaminoglycans or mucopolysaccharides). The later two are cumulatively known as extracellular matrix.

There are three kinds of cartilage, namely hyaline cartilage, elastic cartilage and fibrocartilage. Hyaline cartilage found primarily in joints has a frosted glass appearance with interstitial substance containing fine type II collagen fibers obscured by proteoglycan. Elastic cartilage is a cartilage in which, in addition to the collagen fibers and proteoglycan, the cells are surrounded by a capsular matrix surrounded by an interstitial matrix containing elastic fiber network. The elastic cartilage is found, for example, in the central portion of the epiglottis. Fibrocartilage contains Type I collagen fibers and is typically found in transitional tissues between tendons, ligaments or bones.

The articular cartilage of the joints, such as the knee cartilage, is the hyaline cartilage which consists of approximately 5% of chondrocytes (total volume) seeded in approximately 95% extracellular matrix (total volume). The extracellular matrix contains a variety of macromolecules, including collagen and proteoglycan. The structure of the hyaline cartilage matrix allows it to reasonably well absorb shock and withstand shearing and compression forces. Normal hyaline cartilage has also an extremely low coefficient of friction at the articular surface.

Healthy hyaline cartilage has a contiguous consistency without any lesions, tears, cracks, ruptures, holes or shredded surface. Due to trauma, injury, disease such as osteoarthritis, or aging, however, the contiguous surface of the cartilage is disturbed and the cartilage surface shows cracks, tears, ruptures, holes or shredded surface resulting in cartilage lesions. Partly because hyaline cartilage is avascular, the spontaneous healing of large defects is not believed to occur in humans and other mammals and the articular cartilage has thus only a limited, if any, capacity for repair.

A variety of surgical procedures have been developed and used in attempts to repair damaged cartilage. These procedures are performed with the intent of allowing bone marrow cells to infiltrate the defect and promote its healing. Generally, these procedures are only partly successful. More often than not, these procedures result in formation of a fibrous cartilage tissue (fibrocartilage) which does fill and repair the cartilage lesion but, because it is qualitatively different being made of Type I collagen fibers, it is less durable and less resilient than the normal articular (hyaline) cartilage and thus has only a limited ability to withstand shock and shearing forces than does healthy hyaline cartilage. Since all diarthroid joints, particularly knees joints, are constantly subjected to relatively large loads and shearing forces, replacement of the healthy hyaline cartilage with fibrocartilage does not result in complete tissue repair and functional recovery.

ii. Neocartilage

Neocartilage is an immature hyaline cartilage where the ratio of extracellular matrix to chondrocytes is lower than in mature hyaline cartilage. Mature hyaline cartilage has the ratio of the extracellular matrix to chondrocytes approximately 95:5. The neocartilage has a lower ratio of the extracellular matrix to chondrocytes than mature cartilage and thus comprises more than 5% of chondrocytes.

### B. Differentiation of Universal Chondrocytes

Cells suitable for use in systems and methods of the invention to prepare neocartilage include universal cells. Universal as an adjective as used herein to describe a cell means a cell that has been differentiated from a multi-potent (pluri- or toti-potent) stem cell as a result of human intervention. An illustrative example of universal chondrocytes would be produced by purchasing human pluripotent stem cells from a source such as ATCC (Manassas, Va.) and using laboratory equipment to introduce a growth factor such as TGF-β1 into those human pluripotent stem cells under appropriate culture conditions. Those human pluripotent stem cells would then differentiate into universal chondrocytes. Universal cells may include, for example, chondrocytes, fibroblasts, fibrochondrocytes, tenocytes, osteoblasts, others, or a combination thereof. Stems cells suitable for use in systems and methods of the invention include adult stem cells, mesenchymal stem cells, peripheral blood stem cells, induced pluripotent stem cells, or any combination thereof.

In certain embodiments, neocartilage prepared according to the current invention is grown ex vivo with universal chondrocytes being differentiated from stem cells. Typical sources include cells such as mesenchymal stem cells, induced pluripotent stem cells, or any other type of multipotent stem cells.

Stem cells such as bone marrow-derived mesenchymal stem cells may be induced to differentiate into chondrocytes under specific culture conditions. These conditions include three-dimensional conformation of the cells in aggregates where high cell density and cell-cell interaction play an important role in the mechanism of chondrogenesis. Together with these physical culture conditions, a defined culture medium containing TGF-β1 is useful to achieve chondrogenic differentiation. See Johnstone et al., 1998, In vitro chondrogenesis of bone marrow-derived mesenchymal progenitor cells. Exp Cell Res 238(1):265-72 and Yoo et al., 1998, The chondrogenic potential of human bone-marrow-derived mesenchymal progenitor cells. J Bone Joint Surg Am 80(12):1745-57, incorporated by reference.

Briefly, harvested cells are centrifuged in a benchtop centrifuge at 500×g for 5 min. The cells are resuspended at a density of 1.25×10̂6 cells/ml in chondrogenic differentiation medium. Aliquots of the cell suspension are pipetted (2.5×10̂5 cells/well) into polypropylene 96-well plates and spun in a benchtop centrifuge at 500×g for 5 min then incubated at 37° C. in a humidified atmosphere of 95% air and 5% CO2 for up to 3 weeks. Aspirate the aggregates periodically with medium and medium about every other day. For additional background see Solchaga et al., 2011, Chondrogenic differentiation of bone marrow-derived mesencymal stem cells: tips and tricks, Methods Mol biol 698:253-278, incorporated by reference.

Those culture conditions have been found to work well for larger-scale bioreactor-based tissue engineering. Those methods allow a high-throughput approach to chondrogenic cultures, which reduces both the cost and time with no detrimental effects on the histological and histochemical qualities of the aggregates.

The universal chondrocytes may be further expanded by any method suitable for such purposes such as, for example, by incubation in a suitable growth medium, for a period of several days, typically from about 3 to about 45 days, preferably for 14 days, at about 37° C. Any kind of culture or incubation apparatus or chamber may be used for expanding chondrocytes. The expansion of the cells is preferably associated with the removal of dead chondrocytes, residual native extracellular matrix and other cellular debris before the chondrocytes are selected for culturing and multiplying. Selected chondrocytes are collected and isolated using trypsinization process or any other suitable method.

In certain embodiments, mesenchymal stem cells are differentiated into universal chondrocytes and expanded in a two-dimensional (2D) culture. The expansion step provides a desirable chondrocyte cell count for seeding into a scaffold (i.e. support matrix). Preferably, there are enough chondrocytes to support neocartilage growth during three-dimensional culture. Depending on the amount and quality of the tissue, the chondrocytes may be passaged one or more times in order achieve the desirable cell count. The culture medium for the 2D culture may be, for example, human serum (HS) or heat inactivated fetal bovine serum (HIFBS).

According to certain embodiments, a bioactive agent is introduced into the culture medium during the expansion process. The bioactive agent may include a growth factor, a cytokine, a peptide, a matrix remodeling enzyme, a matrix metalloproteinase, an aggrecanase, a cathepsin, demineralized bone powder, calcium phosphate, hydroxyapatite, organoapatite, titanium oxide, poly-L-lactic acid or a copolymer thereof, polyglycolic acid or a copolymer thereof, and a combination thereof. The growth factor may include a fibroblast growth factor (FGF), a bone morphogenetic protein (BMP), insulin growth factor (IGF), transforming growth factor beta (TGF-B), or a combination thereof. In certain embodiments, the bioactive agent is a bone inducing agent (e.g. fibroblast growth factor).

Suitable fibroblast growth factors include, for example, FGF2, FGF4, FGF9, FGF18, or variants thereof (e.g. FGF2v1). In addition, suitable growth factors include, for example, growth factors discussed in U.S. Pat. Nos. 7,288,406, 7,563,769, and U.S. Publication Nos. 2011/0053841 and 2010/0274362. In particular aspects, a growth factor used in embodiments of the invention is an FGF2 variant. In one embodiment, the FGF2 variant used has the asparagine at position 111 replaced by glycine, and the alanine at position 3 and the serine at position 5 replaced by glutamine, and is denoted as FGF2(3,5Q)-N111G.

The presence of growth factors provides greater than a 100 folds increase in cell count growth in 2D cultures after about 2 weeks of culture as compared to 2D cultures without the presence of a growth factor, which provide about a 25 fold increase after about 2 weeks of culture. The use of a growth factor during 2D expansion advantageously allows for a smaller sample to be taken at biopsy and obviates the need to expand the cells past passage 0. In addition, use of growth factors reduces the culture time to 10 days or fewer. Without a growth factor, two-dimensional culture typically runs from about 10 to about 42 days depending on the number of cells elicited from the biopsy tissue.

Once desired cell count is achieved in the 2D culture, the cells may be prepared for suspension. In certain embodiments, one or more growth factors (or other bioactive agents) exposed to the cells are removed (e.g. using a trypsinization process). The removal of a growth factor at this stage may cause the cells to exhibit gene expression levels more similar to cells of natural cartilage when implanted and/or during incubation in a three-dimensional scaffold (i.e. during 3D culturing). Growth factors or other bioactive agents may be removed from the cells using techniques known in the art, for example, removal of growth factors in the presence of phosphate buffered saline (PBS) or trypsinization. See, for example, Schwindt et al., 2009, Effects of FGF-2 and EGF removal on the differentiation of mouse neural precursor cells, An. Acad. Bras. Ciênc. 81(3):443-452; Flaumenhaft et al., 1989, Role of extracellular matrix in the action of basic fibroblast growth factor: Matrix as a source of growth factor for long-term stimulation of plasminogen activator production and DNA synthesis, J cellular Phys 140(1):75-81, both incorporated by reference. Expanded chondrocytes are then suspended in a suitable solution and seeded into a support matrix to form a seeded matrix. The seeded matrix is typically processed in a tissue processor.

Following or as part of the expansion, the universal chondrocytes are suspended in any suitable solution, preferably collagen containing solution. For the purposes of this invention such solution is typically a gel, preferably sol-gel transitional solution which changes the state of the solution from liquid sol to solid gel above room temperature. The most preferred such solution is the thermo-reversible gelation hydrogel or a thermo-reversible polymer gel. The thermo-reversible property is important both for immobilization of the chondrocytes within the support matrix and for implanting of the neocartilage construct within the cartilage lesion.

In some embodiments, cells expanded with one or more growth factors are introduced into the suspension while still in the presence of the growth factor (which was exposed to the cells during the expansion stage). Alternatively, growth factors added during the expansion step are subsequently removed prior to suspension. In such embodiment, the cells expanded with growth factors, now removed, can be introduced into the suspension. For both embodiments, the expanded cells can be introduced into any of the suspension solutions discussed below.

A bioactive agent may be introduced into the suspension medium with the expanded cells. If the cells are exposed to bioactive agents in both the expansion stage and the suspension stage, the bioactive agent used for the suspension may be the same or different from the bioactive agent used in the expansion stage.

The bioactive agent may include a growth factor, a cytokine, a peptide, a matrix remodeling enzyme, a matrix metalloproteinase, an aggrecanase, a cathepsin, demineralized bone powder, calcium phosphate, hydroxyapatite, organoapatite, titanium oxide, poly-L-lactic acid or a copolymer thereof, polyglycolic acid or a copolymer thereof, and a combination thereof. The growth factor may include a fibroblast growth factor (FGF), a bone morphogenic protein (BMP), insulin growth factor (IGF), transforming growth factor beta (TGF-B), or a combination thereof. In certain embodiments, the bioactive agent is a bone inducing agent (e.g. FGF). Suitable fibroblast growth factors include, for example, FGF2, FGF4, FGF9, FGF18, or variants thereof (e.g. FGF2v1). In addition, suitable growth factors include, for example, growth factors discussed in U.S. Pat. No. 7,288,406; U.S. Pat. No. 7,563,769; U.S. Pub. 2011/0053841; and U.S. Pub. 2010/0274362, all incorporated by reference. In particular embodiments, a growth factor for use in the solution according is an FGF2 variant. In one embodiment, the FGF-2 variant used has the asparagine at position 111 replaced by glycine, and the alanine at position 3 and the serine at position 5 replaced by glutamine, and is denoted as FGF2(3,5Q)-N111G.

One characteristic of the sol-gel is its ability to be cured or transitioned from a liquid into a solid form. This property may be advantageously used for solidifying the suspension of chondrocytes within the support matrix for delivery, storing or preservation purposes. Additionally, these properties of sol-gel also permit its use as a support matrix by changing its sol-gel transition by increasing or decreasing temperature, as described in greater detail below for thermo-reversible gelation hydrogel, or exposing the sol-gel to various chemical or physical conditions or ultraviolet radiation.

In one embodiment the expanded universal chondrocytes are suspended in a collagenous sol-gel solution before incorporation (seeding) into the support matrix. The sol-gel viscosity permits easy mixing of chondrocytes avoiding need to use shear forces. One example of the suitable sol-gel solution is the type I collagen solution formerly available under trade name VITROGEN from Cohesion Corporation (Palo Alto, Calif.) and available sold under the name NUTACON by Nutacon (Leimuiden, Netherlands) and also sold under the trademark PURECOL by Advanced BioMatrix, Inc. (San Diego, Calif.). A preferred type I collagen solution is a purified pepsin-solubilized bovine collagen dissolved in 0.012 N HCl. Sterile collagen for tissue culture may be additionally obtained from other sources, such as, for example, Collaborative Biomedical (Bedford, Mass.) or MediFly Laboratory (Singapore).

When using a type I collagen solution, the cell density is approximately 5-10×106 cells/mL. However, both the density of the cells, the volume for their seeding and strength of the solution are variables within the algorithm, and the higher or lower number of chondrocytes may be suspended in a larger or lower volume of the suspension solution, depending on the size of the support matrix and the size of the cartilage lesion.

Seeding of the suspended chondrocytes into the support matrix is by any means which permit even distribution of the chondrocytes within said support matrix. Seeding may be achieved by bringing the suspension and the support matrix into close contact and seeding the cells by wicking or suction of the suspension into the matrix by capillary action, by inserting the support matrix into the suspension, by using suction, positive or negative pressure, injection or any other means which will result in even distribution of the chondrocytes within said support matrix.

In alternative embodiment, the universal chondrocytes are suspended in the thermo-reversible gelation hydrogel or gel polymer at temperature between 5 and 15° C. At that temperature, the hydrogel is at a liquid sol stage and easily permits the chondrocytes to be suspended in the sol. Once the chondrocytes are evenly distributed within the sol, the sol is subjected to higher temperature of about 30-37° C. at which temperature, the liquid sol solidifies into solid gel having evenly distributed chondrocytes within. The gelling time is from about several minutes to several hours, typically about 1 hour. In such an instance, the solidified gel may itself become and be used as a support matrix or the suspension in sol state may be loaded into a separate support matrix, such as a sponge or honeycomb support matrix.

Other means of generating suspending gels, not necessarily thermo-reversible, are also available and suitable for use. Polyethylene glycol (PEG) derivatives, in which one PEG chain contains vinyl sulfone or acrylate end groups, and the other PEG chain contains free thiol groups will covalently bond to form thio-ether linkages. If one or both partner PEG molecules are branched (three- or four-armed), the coupling results in a network, or gel. If the molecular weight of the PEG chains is several thousand Daltons (500 to 10,000 Daltons along any linear chain segment), the network will be open, swellable by water, and compatible with living cells. The coupling reaction can be accomplished by preparing 5 to 20% (w/v) solutions of each PEG separately in aqueous buffers or cell culture media. Chondrocytes can be added to the thiol-PEG solution. Just prior to incorporation into the support matrix, the cells plus thiol PEG and the acrylate or vinyl sulfone PEG are mixed and infused into the matrix. Gelation will begin spontaneously in 1 to 5 minutes; the rate of gelation can be modulated somewhat by the concentration of PEG reagent and by pH. The rate of coupling is faster at pH 7.8 than at pH 6.9. Such gels are not degradable unless additional ester or labile linkages are incorporated into the chain. Such PEG reagents may be purchased from Shearwater Polymers, Huntsville, Ala., USA; or from SunBio, Korea.

In a second alternative, alginate solutions can be gelled in the presence of calcium ions. This reaction has been employed for many years to suspend cells in gels or micro-capsules. Cells can be mixed with a 1-2% (w/v) solution of alginate in culture media devoid of calcium or other divalent ions, and infused into the support matrix. The matrix can then be immersed in a solution containing calcium chloride, which will diffuse into the matrix and gel the alginate, trapping and supporting the cells. Analogous reactions can be accomplished with other polymers which bear negatively charged carboxyl groups, such as hyaluronic acid. Viscous solutions of hyaluronic acid can be used to suspend cells and gelled by diffusion of ferric ions.

Suspension loaded into the support matrix or gelled into the solid support is processed using the algorithm of the invention. Such processing is performed in a processing apparatus, such as a TESS processor.

### C. Preparation of Support Matrix for Neocartilage Constructs

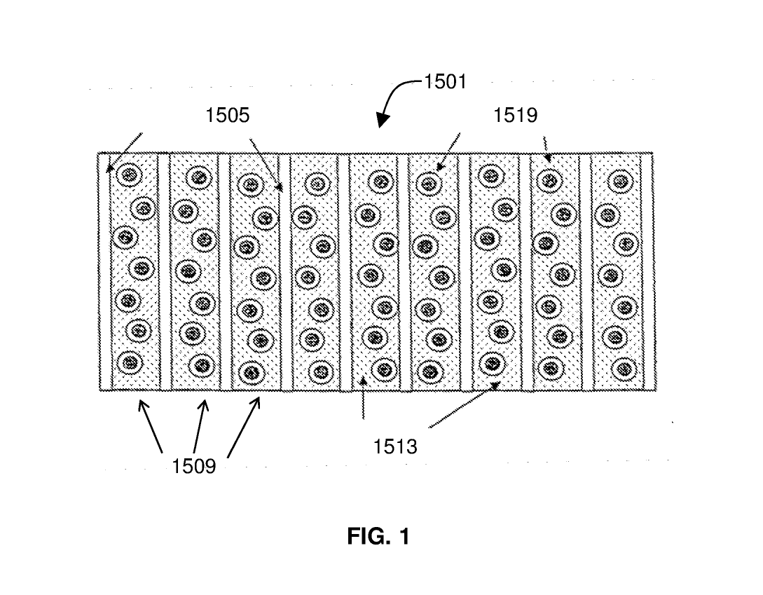

FIG. 1 depicts a composition for cartilage repair in cross-sectional view. The composition includes a bulk implant material 1501 comprising a porous primary scaffold 1505 comprising collagen and a plurality of pores 1509. The bulk implant material 1501 provides a structural support for growth of cells 1519. Generally, the primary scaffold 1505 is biocompatible, hydrophilic and has preferably a neutral charge. Typically, the primary scaffold 1505 is a two or three-dimensional structural composition containing a network of interconnected pores 1509. In some embodiments the primary scaffold 1505 is a sponge-like structure or honeycomb-like lattice.

The bulk implant material 1501 further includes a secondary scaffold 1513 comprising a second collagen disposed within the plurality of pores 1509 and a plurality of living cells 1519 from a universal cell line disposed within the bulk implant material 1501. The bulk implant material is configured such that at least a first cartilage repair implant 1525, a second cartilage repair implant 1525, and a third cartilage repair implant 1527 for a plurality of different human patients may be excised from the bulk implant material. Preferably the bulk implant material is configured such that each of the plurality of different cartilage repair implants may be at least as large as a disc with a diameter of 5 mm and a thickness of 2 mm. In preferred embodiments, the bulk implant material is configured such that each of the plurality of different cartilage repair implants is sized so that it may still be trimmed to yield a final product that is at least about 2 mm thick and has an area of at least about 2.5 cm̂2.

In general, any polymeric material can serve as the primary scaffold 1505, provided it is biocompatible with tissue and possesses the required geometry. Polymers, natural or synthetic, which can be induced to undergo formation of fibers or coacervates, can then be freeze-dried as aqueous dispersions to form sponges. Typically, such sponges are be stabilized by crosslinking. Practical example includes preparation of freeze-dried sponges of poly-hydroxyethyl-methacrylate (pHEMA), optionally having additional molecules, such as gelatin, entrapped within. Such types of sponges can function as support matrices. Incorporation of agarose, hyaluronic acid, or other bio-active polymers can be used to modulate cellular responses. A wide range of polymers may be suitable for the support matrix sponges, including agarose, hyaluronic acid, alginic acid, dextrans, polyHEMA, and poly-vinyl alcohol above or in combination.

Typically, the primary scaffold 1505 is prepared from a collagenous gel or gel solution containing Type I collagen, Type II collagen, Type IV collagen, gelatin, agarose, hyaluronin, cell-contracted collagens containing proteoglycans, glycosaminoglycans or glycoproteins, fibronectin, laminin, bioactive peptide growth factors, cytokines, elastin, fibrin, synthetic polymeric fibers made of poly-acids such as polylactic, polyglycotic or polyamino acids, polycaprolactones, polyamino acids, polypeptide gel, copolymers thereof and combinations thereof. Preferably, the support matrix is a gel solution, most preferably containing aqueous Type I collagen or a polymeric, preferably thermo-reversible, gel matrix.

In some embodiments, a bioactive agent is introduced into the collagenous gel or gel solution used to prepare the primary scaffold 1505. The bioactive agent may include a growth factor, a cytokine, a peptide, a matrix remodeling enzyme, a matrix metalloproteinase, an aggrecanase, a cathepsin, demineralized bone powder, calcium phosphate, hydroxyapatite, organoapatite, titanium oxide, poly-L-lactic acid or a copolymer thereof, polyglycolic acid or a copolymer thereof, and a combination thereof. The growth factor may include a fibroblast growth factor (FGF), a bone morphogenic protein (BMP), insulin growth factor (IGF), transforming growth factor beta (TGF-B), or a combination thereof. In certain embodiments, the bioactive agent is a bone-inducing agent (e.g. fibroblast growth factor) such as, for example, FGF2, FGF4, FGF9, FGF18, or variants thereof (e.g. FGF2v1). Suitable growth factors are discussed in U.S. Pat. No. 7,288,406; U.S. Pat. No. 7,563,769; U.S. Pub. 2011/0053841; and U.S. Pub. 2010/0274362, each incorporated by reference. An FGF2 variant may be used. In one embodiment, the FGF-2 variant used has the asparagine at position 111 replaced by glycine, and the alanine at position 3 and the serine at position 5 replaced by glutamine, and is denoted as FGF2(3,5Q)-N111G.

The gel or gel solution used for preparation of the primary scaffold 1505 is typically washed with water and subsequently freeze-dried or lyophilized to yield a sponge-like matrix able to incorporate or wick the chondrocyte suspension into the matrix. The scaffold may be lyophilized so that it acts like a sponge when infiltrated with the chondrocyte suspension. The resulting scaffold may be implanted into a cartilage lesion. Alternatively, a cell suspension is introduced into the resulting lyophilized scaffold and subject to a three-dimensional culture ex vivo.

One important aspect of the primary scaffold 1505 is the pore size of the primary scaffold 1505. Support matrices having different pore sizes permit faster or slower infiltration of the chondrocytes into said matrix, faster or slower growth and propagation of the cells and, ultimately, the higher or lower density of the cells in the neocartilage construct. Such pore size may be adjusted by varying the pH of the gel solution, collagen concentration, lyophilization conditions, etc. Typically, the pore size of the support matrix is from about 50 to about 500 μm, preferably the pore size is between 100 and 300 μm and most preferably about 200 μm.

The primary scaffold 1505 may be prepared according to procedures described in Example 3, or by any other procedure, such as, for example, procedures described in the U.S. Pat. Nos. 6,022,744; 5,206,028; 5,656,492; 4,522,753; and 6,080,194 herein incorporated by reference.

One preferred type of support matrix is Type-I collagen support matrix fabricated into a sponge, commercially available from Koken Company, Ltd., Tokyo, Japan, under the trade name Honeycomb Sponge.

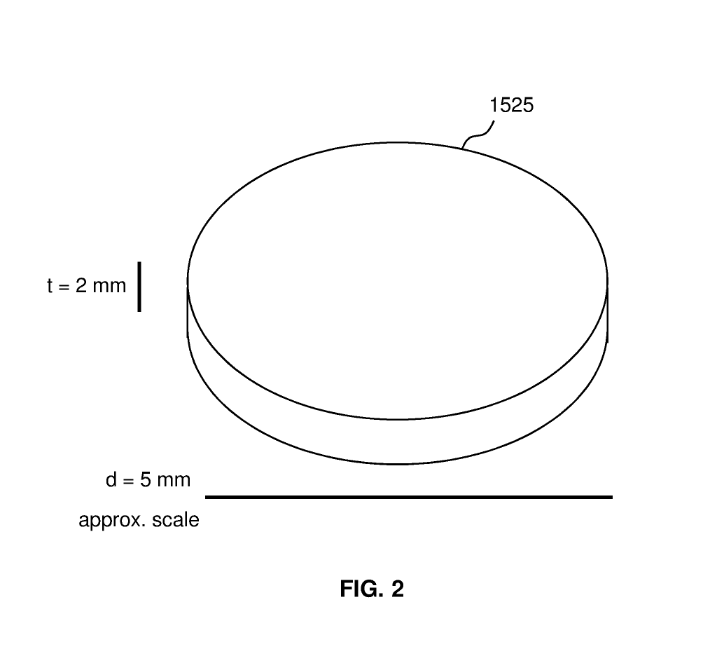

FIG. 2 shows a drawing of a neocartilage construct for use as a cartilage repair implant 1525 having 4 mm in diameter and thickness of 1.5 mm. The seeding density of this construct is 300,000-375,000 chondrocytes per 25 μl of collagen solution corresponding to about 12-15 millions cells/mL. The cell density range for seeding is preferably from about 3 to about 60 millions/mL.

i. Honeycomb Cellular Support Matrix

In one embodiment of the invention, the primary scaffold 1505 is a honeycomb-like lattice matrix providing a cellular support for activated chondrocytes, herein described as neocartilage.

The honeycomb-like primary scaffold 1505 supports a growth platform for the neocartilage and permits three-dimensional propagation of the neocartilage.

The honeycomb-like matrix is fabricated from a polymerous compound, such as collagen, gelatin, Type I collagen, Type II collagen or any other polymer having a desirable properties. In the preferred embodiment, the honeycomb-like matrix is prepared from a solution comprising Type I collagen.

The pores of the honeycomb-like matrix are evenly distributed within said matrix to form a sponge-like structure able to taking in and evenly distributing the neocartilage suspended in a viscous solution.

ii. Sol-Gel Cellular Support Matrix

In another embodiment, the primary scaffold 1505 is fabricated from sol-gel materials wherein said sol-gel materials can be converted from sol to gel and vice versa by changing temperature. For these materials the sol-gel transition occurs on the opposite temperature cycle of agar and gelatin gels. Thus, in these materials the sol is converted to a solid gel at a higher temperature. Sol-gel material is a material which is a viscous sol at temperatures of below 15° and a solid gel at temperatures around and above 37°. Typically, these materials change their form from sol to gel by transition at temperatures between about 15° and 37° and are in transitional state at temperatures between 15° C. and 37°. The most preferred materials are Type I collagen containing gels and a thermo-reversible gelation hydrogel (TRGH) which has a rapid gelation point.

In one embodiment, the sol-gel material is substantially composed of Type I collagen solution (in the form of 99.9% pure pepsin-solubilized bovine dermal collagen dissolved in 0.012N HCl). One important characteristic of this sol-gel is its ability to be cured by transition into a solid gel form wherein said gel cannot be mixed or poured or otherwise disturbed thereby forming a solid structure containing immobilized chondrocytes.

Type I collagen sol-gel is generally suitable for suspending the chondrocytes and for seeding them into a separately prepared support matrix in the sol form and gel the sol into the solid gel by heating the support matrix to a proper temperature, usually around 30-37° and, in this form, processing the embedded support matrix. This type of sol-gel can also be used as a support matrix for purposes of processing the gel containing chondrocytes in the processor of the invention into a neocartilage construct.

In another embodiment, the sol-gel is thermo-reversible gelation hydrogel (TRGH). Sol-gel thermo-reversible material for preparation of sol-gel support matrix is a material which is a viscous sol at temperatures of below 15-30° C. and solid gel at temperatures above 30-37° C. The primary characteristic of the thermo-reversible gelation hydrogel (TRGH) is that it gels at body temperature and sols at lower than 15-30° C. temperature, that upon its degradation within the body it does not leave biologically deleterious material and that it does not absorb water at gel temperatures. TRGH has a very quick sol-gel transformation which requires no cure time and occurs simply as a function of temperature without hysteresis. The sol-gel transition temperature can be set at any temperature in the range from 5° C. to 70° C. by the molecular design of the thermo-reversible gelation polymer (TGP), a high molecular weight polymer of which less than 5 wt % is enough for hydrogel formation.

The typical TRGH is generally made of blocks of high molecular weight polymer comprising numerous hydrophobic domains cross-linked with hydrophilic polymer blocks. TRGH has low osmotic pressure and is very stable as it is not dissolved in water when the temperature is maintained above the sol-gel transition temperature. Hydrophilic polymer blocks in the hydrogel prevent macroscopic phase separation and separation of water from hydrogel during gelation. These properties make it especially suitable for safe storing and extended shelf-life.

The thermo-reversible gelation hydrogel (TRGH), particularly a space-holding thermo-reversible gel (SHTG), should be a compressively strong and stable at 37° C. and below till about 32° C., that is to about temperature of the synovial capsule of the joint which is typically below 37° C., but should easily solubilize below 30-31° C. to be able to be conveniently removed from the cavity as the sol. The compressive strength of the SHTG or TRGH must be able to resist compression by the normal activity of the joint.

In this regard, the thermo-reversible hydrogel is an aqueous solution of thermo-reversible gelation polymer (TGP) which turns into hydrogel upon heating and liquefies upon cooling. TGP is a block copolymer composed of temperature responsive polymer (TRP) block, such as poly(N-isopropylacrylamide) or polypropylene oxide and of hydrophilic polymer blocks such as polyethylene oxide.

Thermally reversible hydrogels consisting of co-polymers of polyethylene oxide and polypropylene oxide are available from BASF Wyandotte Chemical Corporation under the trade name of Pluronics.

In general, thermo-reversibility is due to the presence of hydrophobic and hydrophilic groups on the same polymer chain, such as in the case of collagen and copolymers of polyethylene oxide and polypropylene oxide. When the polymer solution is warmed, hydrophobic interactions cause chain association and gelation; when the polymer solution is cooled, the hydrophobic interaction disappears and the polymer chains are dis-associated, leading to dissolution of the gel. Any suitably biocompatible polymer, natural or synthetic, with such characteristics will exhibit the same reversible gelling behavior.

This type of thermo-reversible gelation hydrogel is particularly preferred for preparation of neocartilage constructs for implantation of the construct into the lesion. In such an instance, the harvested chondrocytes are suspended in the TRGH sol, then warmed to about 37° C. into the solid gel which thus itself becomes a seeded support matrix, then submitting said seeded matrix to the processing in the tissue processor using the algorithm of the invention, including resting period as described below, thereby resulting in a formation of the neocartilage construct, then submitting said construct to cooling to change its form into a sol and in this form injecting the neocartilage into the lesion wherein upon warming to body temperature the sol is immediately converted into the gel containing neocartilage. In time, the delivered neocartilage is integrated into the existing cartilage and the TRGH is subsequently degraded leaving no undesirable debris behind.

iii. Scaffold with Fibrous Collagen Network

In certain aspects, a construct of the invention includes a porous primary scaffold 1505 having a fibrous-collagen secondary scaffold 1513 dispersed within and spanning across the pores of the scaffold. In order to create the fibrous-collagen secondary scaffold 1513, a solution is disposed and then stabilized within the pores. The solution may be added to any of the cellular support matrices described herein. The solution may be used to generate a fibrous collagen secondary scaffold 1513 within the pores. The collagen fibers interdigitate within and across the pores. The collagen network formed within the pores adds additional support to the matrix and provides more surface for the chondrocytes to expand into and develop the extracellular matrix. The solution for forming the fibrous collagen secondary scaffold 1513 may be a soluble collagen-based composition. In certain embodiments, solution further includes a suitable surfactant (basic solution). Scaffolds with fibrous-collagen secondary scaffold 1513 suitable for use in constructs and methods of the invention are described in more detail in co-owned U.S. Pub No. 2009/001267, incorporated by reference.

The solution for the fibrous collagen secondary scaffold 1513 may include a collagen, collagen-containing and collagen-like mixtures, said collagen being typically of Type I or Type II, each alone, in a mixture, or in combination. The solution may also include a surfactant, preferably a non-ionic surfactant, in combination with the collagen, methylated collagen, gelatin or methylated gelatin, collagen-containing and collagen-like mixtures. Typically, the surfactant is a non-ionic surfactant.

Suitable surfactants include non-ionic co-polymer surfactants consisting of polyethylene and polypropylene oxide blocks. Suitable surfactants may include commercially available derivatized polyethylene oxides, such as for example, polyethylene oxide p-(1,1,3,3-tetramethylbutyl)-phenyl ether, known under its trade name as TRITON-X100. Other suitable surfactants include commercially available block co-polymers of polyoxyethylene (PEO) and polyoxypropylene (PPO) having the following generic organization of polymeric blocks: PEO-PPO-PEO (Pluronic) or PPO-PEO-PPO (Pluronic R). A preferred non-ionic surfactant for use in the invention is a block co-polymer of polyoxyethylene (PEO) and polyoxypropylene (PPO) with two 96-unit hydrophilic PEO blocks surrounding one 69-unit hydrophobic PPO block, known under its trade name as PLURONIC F127 commercially available from BASF Corp.

After generation of the porous primary scaffold 1505, the solution for the fibrous collagen secondary scaffold 1513 may be added. In one embodiment, the solution is added to the porous primary scaffold 1505 by soaking or immersing the porous primary scaffold 1505 in the solution. In addition, the solution may be added to the porous primary scaffold 1505 by absorbing, wicking, or by using a pressure, vacuum, pumping or electrophoresis, etc. In alternative, the porous primary scaffold 1505 may be immersed into the solution for forming the fibrous collagen network.

The solution for the fibrous secondary scaffold 1513 may include a bioactive agent. The bioactive agent may include a growth factor such as is discussed above, a cytokine, a peptide, a matrix remodeling enzyme, a matrix metalloproteinase, an aggrecanase, a cathepsin, demineralized bone powder, calcium phosphate, hydroxyapatite, organoapatite, titanium oxide, poly-L-lactic acid or a copolymer thereof, polyglycolic acid or a copolymer thereof, and a combination thereof. Once the solution is exposed to the support matrix, the combined scaffold and solution is precipitated or gelled, washed, dried, lyophilized and dehydro-thermally treated to solidify and stabilize the solution within the pores of the support matrix. Once stabilized, the solution forms a fibrous collagen network as a secondary structure within the pores of the scaffold.

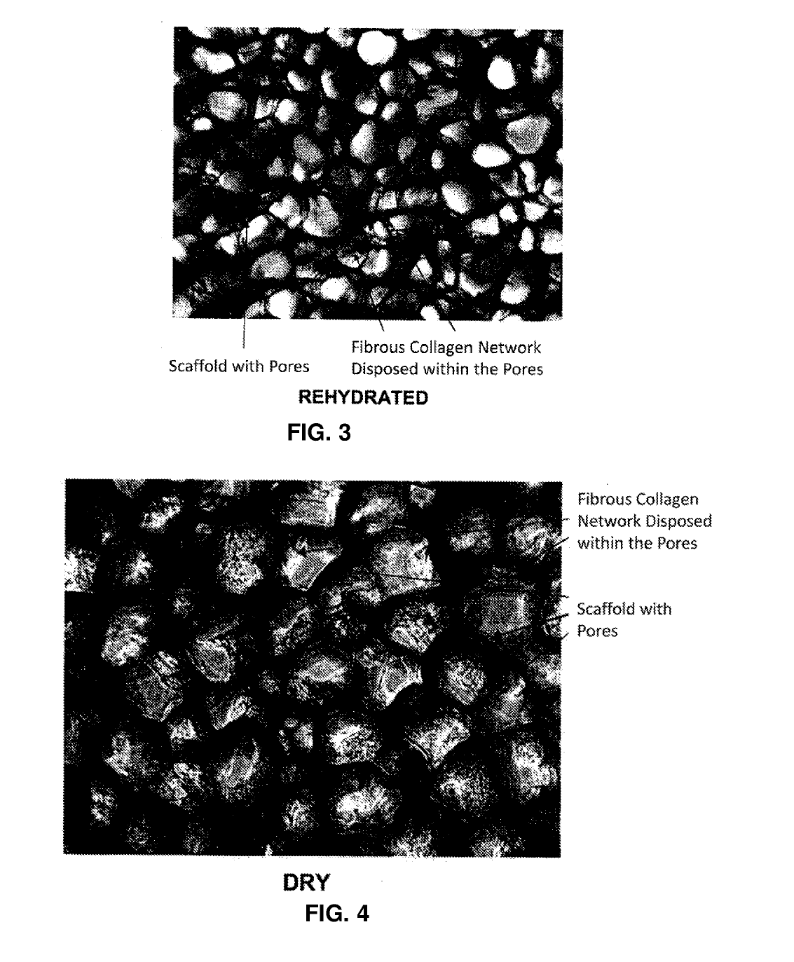

FIG. 3 shows a rehydrated double-structured tissue implant in which the secondary scaffold 1513 is observed from the fibrous-like diffraction pattern present within the pores of the primary scaffold 1505. The diffraction pattern occurs due to the polymerization of the collagen within the pores. The collagen fibers interdigitate within the pores and among the pores.

FIG. 4 shows a dry form of the double-structured tissue implant.

The stabilized support matrix/solution system may be directly implanted into the cartilage lesion for repair. Alternatively, the support matrix/solution system may be loaded with a cell suspension as described above and subject to three-dimensional culture ex vivo using a method described below.

### D. Processing Neocartilage and Tissue Processors

In order to promote three-dimensional growth and propagation of universal chondrocytes and/or neocartilage, it may be beneficial to facilitate such growth and propagation by changing conditions of their growth. This may include subjecting either the suspended universal chondrocytes or the support matrix incorporated with suspended chondrocytes to certain conditions which were found to promote such propagation. Such conditions are, for example, application of constant or cyclic hydrostatic pressure, resting periods at static pressure, recirculation and changing flow rate of media, regulation of oxygen or carbon dioxide concentrations, cell density, control pH, availability of nutrients and co-factors, etc. Typically, this process is performed in the tissue processor, permitting changing of the conditions, as stated above. In certain embodiments, three-dimensional culture conditions do not require cyclic hydrostatic pressure. For example, a hydrostatic pressure may not be applied when the cells are treated with a bioactive agent in the expansion or suspension steps, or when the support matrix has been treated with a bioactive agent. In particular embodiments, cells that were expanded in the presence of a growth factor or other bioactive agent are subject to three-dimensional culture conditions without application of a mechanical stimulus, as such bioactive agent was removed prior to suspension and introduction of the cell suspension in the scaffold.

i. Neocartilage Tissue Processor

The general design of the tissue processor is the apparatus for culturing chondrocytes comprising a culture unit having a culture chamber containing culture medium and a supply unit for the continuous and intermittent delivery of the culture medium, a pressure generator for applying atmospheric or constant or cyclic hydrostatic pressure above the atmospheric pressure to chondrocytes in the tissue chamber, said generator having means for changing the pressure, timing, or applying the atmospheric, constant or cyclic hydrostatic pressure at predetermined periods and, optionally, a means capable of delivering and/or absorbing gases such as nitrogen, carbon dioxide and oxygen. Additionally, the processor typically comprises a hermetically sealed space including a heating, cooling and humidifying means.

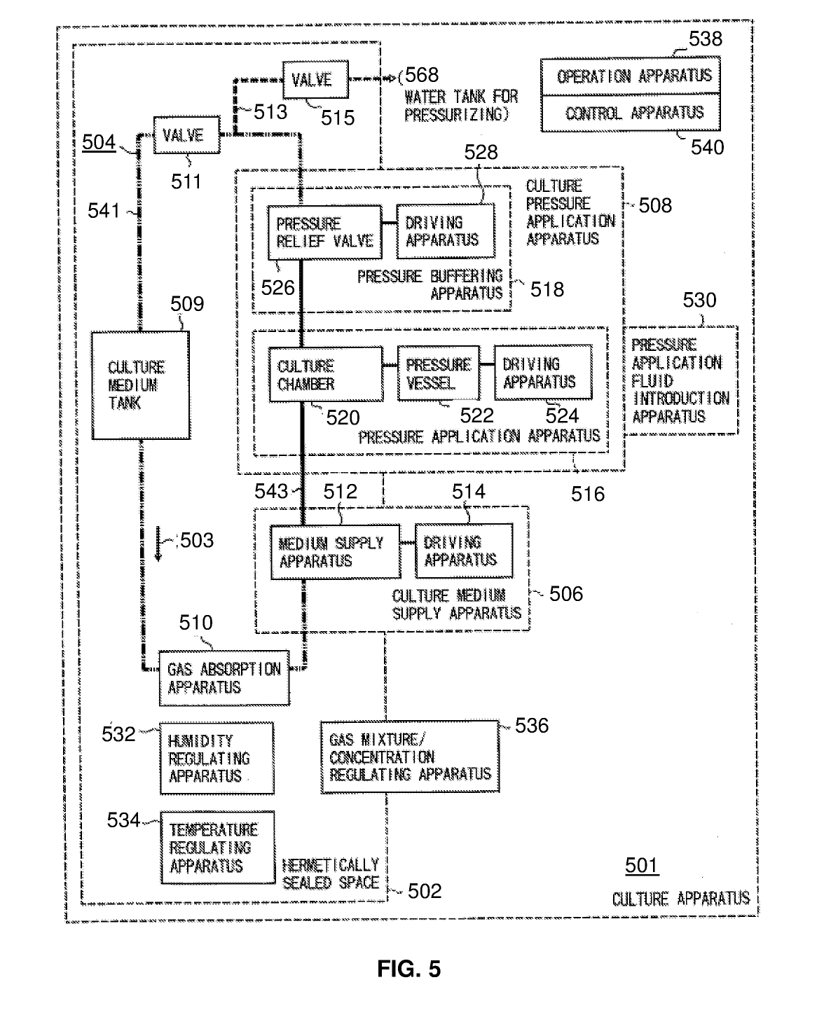

FIG. 5 gives a diagram of a tissue processor system suitable for applying of static or hydrostatic pressure, changing flow rate of the medium and regulating gas concentration delivered to the embedded tissue engineering support system. A culture apparatus 501 for realizing the method of cultivating tissue has a hermetically sealed space 502 as a culture space in which a culture circuit unit 504 serving as a culture unit to supply culture medium 503 to tissue to be cultivated is installed.

The culture circuit unit 504 can be set up so as to be separated or detachable from a body of the culture apparatus 501 (hereinafter referred to as culture apparatus body). The culture circuit unit 504 includes a culture medium tank 509, culture medium supply apparatus 50, a culture pressure application apparatus 508, a gas absorption apparatus 510, a valve 511, and a branched path 513 having a valve 515 thereon. The culture medium 503 is a carrier for supplying a nutrition to the tissue to be cultivated and a fluid including essential amino acid and various amino acids, glucose (saccharide), and an sometimes inorganic material such as Na+, Ca++ is added thereto depending on the cell or tissue to be cultivated or a protein such as serum is included therein. Further, these apparatus are formed of a resin material having a sufficient heat resistance and does not melt to produce a material that affects a living body such as a fluorine resin, PEEK, a high grade heat resistant polypropylene, silicone or stainless steel, thereby preventing the constituents from being contaminated.

The valves 511, 515 may be formed of a pinch valve and so forth. The culture circuit unit 504 forms a closed loop circuit when the valve 515 is shut and the valve 511 is opened, an entire open loop circuit when the valve 515 is opened and the valve 511 is shut, and a partial open loop circuit when both the valves 511, 515 are opened. The culture circuit unit 504 may include a gas absorption portion 541 denoted by two dotted one chain line and a pressure resistant portion 543 denoted by a solid line instead of the gas absorption apparatus 510 that is partially installed therein. The gas absorption portion 541 is a portion to render gas filled in the hermetically sealed space 502 to be absorbed by the culture medium 503 while the pressure resistant portion 543 is a portion to assure a reliable medium supply, corresponding to the pressure application portion of the culture medium 503 so as to prevent leakage of medium. A tube formed of an elastomer material through which gas easily passes a gas such as CO₂, O₂ may be used in the gas absorption portion 541.

The culture medium tank 509 is accommodated in the hermetically sealed space 502 and means for storing therein the culture medium 503 that is needed for cultivating the cell or tissue. The culture medium supply apparatus 506 is means for supplying the culture medium 503 to the culture circuit unit 504, namely, when a medium supply apparatus 512 that is inserted into the culture circuit unit 504 is driven by a driving apparatus 514, it supplies a predetermined amount of culture medium 503 to the culture circuit unit 504. The culture pressure application apparatus 508 is means for applying a pressure to a tissue to be cultivated, and includes a pressure application apparatus 516 and a pressure buffering apparatus 518. The pressure application apparatus 516 comprises a culture chamber 520 of the culture circuit unit 504, a pressure vessel 522 attached to the culture chamber 520 and a driving apparatus 524 for allowing an arbitrary pressure to act on the culture chamber 520. A cell or tissue to be cultivated is transplanted in a scaffold formed of a collagen and so forth and it is accommodated in the culture chamber 520 and is separated from the outside.

The pressure buffering apparatus 518 is means for buffering a pressure to be applied to the culture medium 503 by the culture pressure application apparatus 508, and it sets a pressure of the culture medium 503 exceeding a predetermined value as the maximum pressure by driving a pressure relief valve 526 that is inserted into the culture circuit unit 504 by a driving apparatus 528. When a pressure of the culture medium 503 exceeding the maximum pressure acts on the culture circuit unit 504, the pressure buffering apparatus pressure 518 operates the pressure relief valve 526 to allow the culture medium 503 to escape therefrom, thereby buffering the pressure. A pressure application fluid is introduced into the pressure vessel 522 from a pressure application fluid introduction apparatus 530 provided together with the culture pressure application apparatus 508.

A humidity regulating apparatus 532, a temperature regulating apparatus 534, and a gas mixture/concentration regulating apparatus 536 are installed in the culture apparatus 501 to regulate an atmospheric humidity, an atmospheric temperature and gas mixture and concentration. An operation apparatus 538 and a control apparatus 540 are respectively installed in the culture apparatus 501, wherein desired control operations are performed by an administrator using the operation apparatus 538 while the control apparatus 540 is means for controlling a various apparatus such as the culture medium supply apparatus 506, culture pressure application apparatus 508, pressure application fluid introduction apparatus 530, humidity regulating apparatus 532, temperature regulating apparatus 534, gas mixture/concentration regulating apparatus 536 in response to an operation input or a control program through the operation apparatus 538.

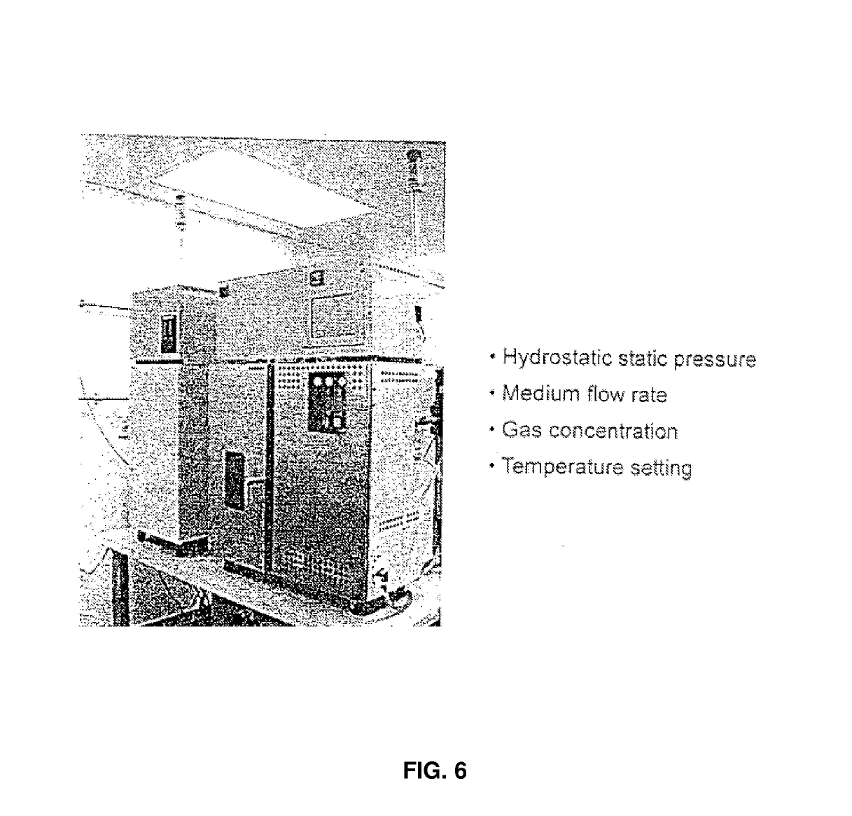

FIG. 6 shows a tissue processor known as Tissue Engineering Support System (TESS) housing the culture apparatus 501. Such systems are described in the U.S. Pat. No. 6,432,713 and in U.S. Pat. No. 6,607,917, both incorporated by reference.

ii. Biochemical and Histological Testing of Neocartilage Constructs

The neocartilage constructs are tested for their metabolic activity, genetic activation and histological appearance. Typically, the constructs are harvested at days 6 and 18. For histological evaluation of the immature and mature cartilage matrix, 4% paraformaldehyde-fixed paraffin sections are stained with Safranin-O and Type II collagen antibody. For biochemical analysis, neocartilage constructs are digested in papain at 60° C. for 18 hours and DNA is measured using, for example, Hoechst 33258 dye method as described in Anal. Biochem 174:168-176 (1988). The production of glycoaminoglycan (GAG) or sulfated-glycosaminoglycan (S-GAG) indicating a metabolic activity of the chondrocyte culture may be tested by a modified dimethylene blue (DMB) microassay according to Connective Tissue Research, 9:247-248 (1982).

iii. Conditions for Propagation of Chondrocytes, Preparation of Neocartilage and Neocartilage Constructs

Neocartilage construct, as used herein, means a matrix embedded with chondrocytes and processed according to the invention. Neocartilage constructs may be produced as 3-dimensional patches comprising neocartilage having an approximate size of the lesion into which they are deposited or they may be produced as 3-dimensional sheet for use in repairs of extensive cartilage injuries. Their size and shape is determined by the shape and size of the support matrix. Their functionality is determined by the conditions (the algorithm) under which they were processed.

Conditions for three-dimensional propagation of chondrocytes in the support matrix into neocartilage construct are variable and are adjusted according to the intended use and/or function of the neocartilage and depend on the type of used thermo-reversible hydrogel and on the density of the seeded cells. Thus for production of small neocartilage constructs, the conditions will be different from those needed for production of large constructs or for production of extensive neocartilage sheets for partial or total replacement of extensively damaged or diseased, for example osteoarthritic, cartilage.

a. Processing Neocartilage Under Variable Flow

One aspect of this invention is the discovery that if the support matrix seeded with chondrocytes is perfused under varying medium flow rates, the cell proliferation, measured by increased accumulation of the extracellular matrix, can be advantageously increased or decreased. Generally, the lower medium flow rate results in the higher extracellular matrix accumulation.

Perfusion is an important variable condition for culturing chondrocytes incorporated into support matrices. Using a faster perfusion flow rate may slow down extracellular matrix accumulation affecting growth and propagation of chondrocytes, as measured by production of sulfated glycosaminoglycan (S-GAG). A slower perfusion rate, on the other hand, results in higher production of S-GAG. These results are important for controlling the neocartilage growth and for, for example, storage, preservation, transport and shelf-life of neocartilage constructs.

The perfusion flow rate suitable for purposes of this invention is from about 1 to about 500 μl/min, preferably from about of 5 to about 50 μl/min. At the medium perfusion rate 5 μl/min the accumulation of extracellular matrix is significantly (p<0.05) increased compared to accumulation of extracellular matrix observed following perfusion at rate 5 μl/min. The optimum flow rate depends upon the total number of cells in the culture chamber.

b. Processing Neocartilage Under Different Types of Pressure

The seeded support matrix may be subject to static (atmospheric pressure), hydrostatic pressure or a combination thereof. Cells exposed to a growth factor (or other bioactive agent) during the expansion step, suspension step, due to a bioactive agent present in the support matrix, or combinations thereof may be subject to static pressure alone, hydrostatic pressure, or cyclic hydrostatic pressure. Different types of hydrostatic pressure have a significant effect on glycosaminoglycan production and thus on extracellular matrix accumulation compared to the effect of atmospheric pressure alone when not treated with a bioactive agent. However, when chondrocytes are introduced to a bioactive agent in accordance to methods of the invention, application of static pressure without application of a mechanical stimulus has been found to stimulate chondrocyte proliferation and metabolism which contributes to extracellular matrix accumulation.

Hydrostatic pressure suitable for processing chondrocytes embedded within the support matrix is either a constant or cyclic hydrostatic pressure, such pressure being the pressure above the atmospheric pressure. The cyclic hydrostatic pressure suitable for use in processing of the seeded support matrix is from about 0.01 to about 10.0 MPa, preferably from about 0.5 to about 5.0 MPa and most preferably at about 3.0 MPa at 0.01 Hz to about 2.0 Hz, preferably at about 0.5 Hz, applied for about 1 hour to about 30 days, preferably about 7 to about 14 days, with or without resting period. Typically, the period of hydrostatic pressure is followed by the resting period, typically from about 1 day to about 60 days, preferably for about 7 to about 28 days, most preferably for about 12 to about 18 days.

Studies performed in support of this invention indicate that cell viability is not affected by the hydrostatic pressure and is maintained with chondrocytes distributed uniformly within the support matrix. Following the treatment with hydrostatic pressure, accumulations of both DNA and S-GAG are significantly increased compared to cultures not experiencing applied load, indicating that chondrocyte activation and metabolic and genetic activity can be controlled by the culture environment. In addition, studies performed in support of this invention indicate that cells exposured to a growth factor during expansion exhibit similar levels of DNA and S-GAG accumulation when treated with static pressure alone or hydrostatic pressure (cyclic or constant).

c. Processing Neocartilage Under Reduced Oxygen Concentration

Another variable in the processing of seeded support matrices is the concentration of oxygen, carbon dioxide and nitrogen. The universal chondrocytes-embedded support matrix described above may be further cultured under reduced 02 concentration (i.e. less than 20% saturation) during formation of neocartilage in the TESS processor. The reduced oxygen concentration of cartilage has been observed in vivo, and such reduction may be due to its normal lack of vascularization which produces a lower oxygen partial pressure, as compared to the adjacent tissues. In this set of studies, chondrocytes seeded in support matrix or neocartilage were cultured under oxygen concentration between about 0% and about 20% saturation or under dioxide concentration about 5%.

### E) Varying Methods for Preparing Neocartilage Constructs

Disclosed are conditions for preparation of neocartilage constructs for implantation into cartilage lesions, which in conjunction with deposition of one or two sealant layers as well as the use of universal chondrocytes, lead to healing of the damaged, injured, diseased or aged cartilage by (a) growth of superficial cartilage layer completely overgrowing and covering the lesion and protecting implanted neocartilage construct; (b) integration of neocartilage implanted into the lesion as the neocartilage construct; and (c) subsequent degradation of the construct and sealant materials.

The following methods are aimed at increasing activation of universal chondrocytes. Increased cell proliferation (dividing and multiplying chondrocytes) shows that the harvested inactive non-dividing chondrocytes have been activated into neocartilage. In addition, increased levels of DNA show genetic activation of inactive chondrocytes. Increased production of Type II collagen and S-GAG is also an indicator that the cells have been activated.

In one embodiment, a method for preparation of neocartilage constructs includes obtaining universal chondrocytes; expanding the chondrocytes for about 3-28 days; seeding chondrocytes in a thermo-reversible or collagen gel or collagen sponge support matrix; subjecting the seeded gel or sponge to a static, constant or cyclic hydrostatic pressure above atmospheric pressure (about 0.5-3.0 MPa at 0.5 Hz) with medium perfusion rate of 5 μl/min for several (5-10) days; and subjecting the seeded gel or sponge to resting period for ten to fourteen days at constant (atmospheric) pressure.

Neocartilage constructs obtained by the above-outlined conditions and method show that the combined algorithm of hydrostatic pressure and static pressure has advantages over conventional culture methods by resulting in higher cell proliferation and extracellular matrix accumulation. Use of thermo-reversible or collagen gel or collagen sponge support matrix maintains uniform cell distribution within the support matrix and also provides support for newly synthesized extracellular matrix. Obtained 3-dimensional neocartilage construct is easy to handle and manipulate and can be easily and safely implanted in a surgical setting.

Combination of a period of cyclic hydrostatic pressure under low medium perfusion rate followed up with a period of static culture (resting period) results in increased cell proliferation, increased production of Type II collagen, increased DNA content and increased S-GAG accumulation.

In another embodiment, a method for preparation of neocartilage constructs includes obtaining universal chondrocytes; expanding the chondrocytes for about 3-28 days in the presence of a growth factor (such as FGF2 or a variant thereof); removing the growth factor from the expanded chondrocytes; seeding chondrocytes in a thermo-reversible or collagen gel or collagen sponge support matrix; subjecting the seeded gel or sponge to a static pressure alone or hydrostatic pressure (cyclic or constant) above atmospheric pressure (about 0.5-3.0 MPa at 0.5 Hz) with medium perfusion rate of 5 μl/min for several (5-10) days; and subjecting the seeded gel or sponge to resting period for ten to fourteen days at constant (atmospheric) pressure.

Neocartilage constructs obtained by the above-outlined conditions and method show that the use of a growth factor during the expansion phase result in higher cell proliferation and extracellular matrix accumulation than cells not treated with a growth factor. That is, cells expanded with FGF2v1 in 2D culture resulted in increased cell proliferation, increased production of Type II collagen, increased DNA content and increased S-GAG accumulation in the subsequent 3D culture.

In another embodiment, a method for preparation of neocartilage constructs includes obtaining universal chondrocytes; expanding the chondrocytes for about 3-28 days; seeding chondrocytes in a thermo-reversible or collagen gel or collagen sponge support matrix, wherein a growth factor (e.g. FGF2 or variants thereof) is introduced during the expansion step or the seeding step (into suspension and/or support matrix); subjecting the seeded gel or sponge to a static pressure or hydrostatic pressure (cyclic or constant) above atmospheric pressure (about 0.5-3.0 MPa at 0.5 Hz) with medium perfusion rate of 5 μl/min for several (5-10) days; and subjecting the seeded gel or sponge to resting period for ten to fourteen days at constant (atmospheric) pressure.

Validation of Culture Conditions

The embodiments described above for chondrocytes is similarly applicable to other types of cell and tissue, such as fibroblasts, fibrochondrocytes, tenocytes, osteoblasts and stem cells capable of differentiation, or tissues such as cartilage connective tissue, fibrocartilage, tendon and bone. The culture conditions may be the same or different but would be generally within the above described ranges.

The underlying studies, described below, show that a properly designed and optimized culture conditions according to certain embodiments of the invention result in fabrication of neocartilage constructs which are integrated into the native cartilage when implanted under the one layer or in between two layers of sealants according to the invention. In addition, the introduction of a growth factor allows chondrocytes to be activated in presence of a static pressure alone or hydrostatic pressure (constant or cyclic) with comparable results.

### F. Supporting Experimental Studies for Application of Hydrostatic Pressure

In order to test effects of different conditions on the propagation of universal chondrocytes within the support matrix for fabrication of the neocartilage construct, studies combining conditions described above for process optimization were performed during development of certain embodiments of this invention. Results are shown in FIGS. 3-9 and in Tables 1-3.

TABLE 1

| | Pressure Conditions | | | |
|---|---|---|---|---|
| | In TESS | | | |
| | (3 MPa Cyclic | In Incubator | Total | S-GAG Production |
| Group | Pressure, | (Atmospheric | days in | (μg/cell construct) |
| (n = 6) | @0.5 Hz) | Pressure) | Culture | (Mean ± SD) |
| Initial | — | 0 day | 0 | 12.56 ± 0.99 |
| Control | — | 18 days | 18 | 57.73 ± 6.43 |
| Test | 6 days | 12 days | 18 | *76.32 ± 4.12 |

(*p < 0.05, Compared to Control)

TABLE 2

| | Pressure Conditions | | | | | |
|---|---|---|---|---|---|---|
| | | | Days in | | S-GAG | |
| | In TESS | | Incubator | Total | GAG Production | DNA |
| Group | Type of | Time/ | (Atmospheric | days In | (μg/cell construct | DNA Index |
| (n = 7) | Pressure | Days | Pressure) | culture | (Mean ± SD) | (Control = 1) |
| Control | — | — | 18 | 18 | 59.85 ± 7.69 | 1 |
| Cy-HP | 0.5 MPa | 6 | 12 | 18 | *91.05 ± 10.68 | 1.49 |
| Cylic | | | | | | |
| Const-HP | 0.5 MPa | 6 | 12 | 18 | *97.85 ± 5.53 | 1.74 |
| Constant | | | | | | |

(*p < 0.05, compared to Control)

For all following studies, the experimental design was as follows with changes in studies conditions.

Cartilage was harvested under sterile conditions from the trachea of the swine hind limbs, minced and digested. Chondrocytes were expanded for 5 days at 37° C. and suspended in type I collagen solution (300,000/30 μl). The suspension was absorbed into a support matrix, usually a collagen sponge (4 mm in diameter and 2 mm in thickness) as seen in FIG. 1, commercially available from Koken Co., LTD (Tokyo, Japan). The sponges seeded with chondrocytes were pre-incubated for 1 hour at 37° C. to gel the collagen, followed by incubation in culture medium at 37° C., 5% CO2 and cultured in the Tissue Engineering Support System (TESS) processor seen in FIG. 6.

i. Evaluation of Effect of Hydrostatic Pressure

To evaluate the effect of the pressure and/or medium perfusion rate, the cell seeded sponges were subjected to medium perfusion at 5 μl/min (0.005 mL/min) or 50 μl/min (0.05 mL/min) under the cyclic (Cy-HP) or constant hydrostatic pressure (constant-HP) of 0.5 MPa at 0.5 Hz for 6 days in the TESS processor. Resting period under atmospheric pressure followed for 12 days. Some seeded sponges served as controls. These were incubated under the atmospheric pressure and without perfusion at 37° C. for a total of 18 days in culture. Sponges harvested 24 hours after seeding with cells (day 0) served as an initial control.

At the end of culture period, the support matrices were harvested for biochemical and histological analysis. Sulfated glycosaminoglycan production was measured using a modified dimethylmethylene blue microassay. Histological analysis utilized Safranin-O staining.

The first study was directed to determination of effect of constant (atmospheric), cyclic or constant hydrostatic pressure on production of S-GAG. At the end of the culture period, both control and test matrices were harvested for biochemical and histological analysis. For biochemical analysis, production of sulfated glycosaminoglycan (S-GAG pg/cell construct) was measured using a modified dimethylmethylene blue (DMB) and DNA microassays. Results are seen in Tables 1 and 2 and FIGS. 3-6.

Results of some studies are seen in Tables 1 and 2 showing a numerical representation of observed increase in S-GAG production in matrices treated with the algorithm of the invention.

Table 1 summarizes results obtained from seeded matrices (n=6) subjected either to atmospheric pressure in an incubator for 18 days (control) or to processing in TESS processor under 3 MPa cyclic hydrostatic pressure at 0.5 Hz for 6 days, followed by 12 days in incubator at atmospheric pressure (test).

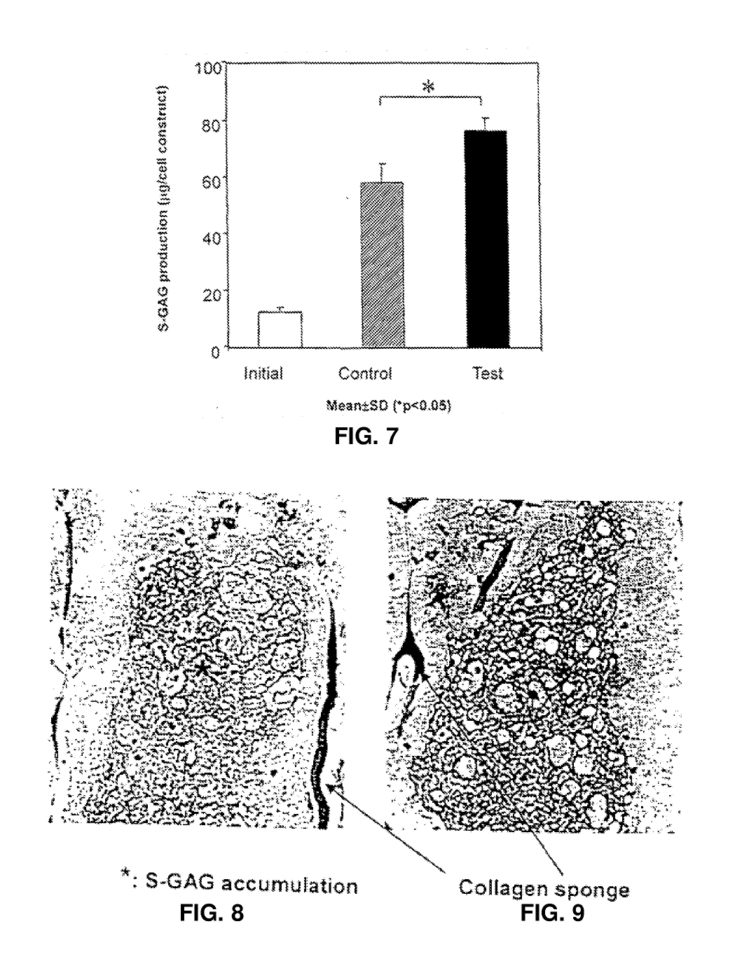

FIG. 7 is a graph illustrating that S-GAG production (μg/cell construct) per seeded matrix was significantly increased to 132% for test compared to 100% control. Histological results seen in FIGS. 8 and 9 (Safranin-O staining for S-GAG) were consistent with the results seen in Table 1 obtained biochemically.

FIG. 8 is a photomicrograph of Safranin-O staining for S-GAG on paraffin sections in 18 days subjected to static pressure. FIG. 9 is a photomicrograph of Safranin-O staining for S-GAG on paraffin sections in cell constructs subjected to cyclic hydrostatic pressure for 6 days followed by 12 days of static culture.

As seen in FIG. 8, when the cell constructs are subjected to static atmospheric pressure (FIG. 8), there is much lower S-GAG accumulation in the constructs than when it is subjected to a cyclic hydrostatic pressure for 6 days, followed by 12 days of static atmospheric pressure (FIG. 9).

To determine the effect of the hydrostatic pressure on chondrocyte proliferation stimulation and matrix accumulation, cartilage was harvested under sterile conditions as described above. Chondrocytes were expanded for 5 days at 37° C. and suspended in type I collagen solution (300,000/30 μl). The suspension was absorbed into a honeycomb support matrix or collagen sponge as seen in FIG. 1. The cell constructs were incubated in culture medium at 37° C., 5% CO. 2 and 20% O₂, at 0.5 MPa cyclic hydrostatic pressure or 0.5 MPa constant hydrostatic pressure for 6 days followed by incubation for 12 days at atmospheric pressure in the Tissue Engineering Support System (TESS) processor seen in FIG. 6. The remaining cell matrices comprising the control group were incubated at atmospheric pressure for 18 days at 37° C., 5% CO₂ and 20% O₂.

At the end of the culture period, the matrices were harvested for biochemical analysis. Glycosaminoglycan production was measures using a modified dimethylmethylene blue (DMB) microassay. Cell proliferation was measured using a modified Hoechst Dye DNA assay. Formation of neo-tissue was evaluated by Safranin-O staining.

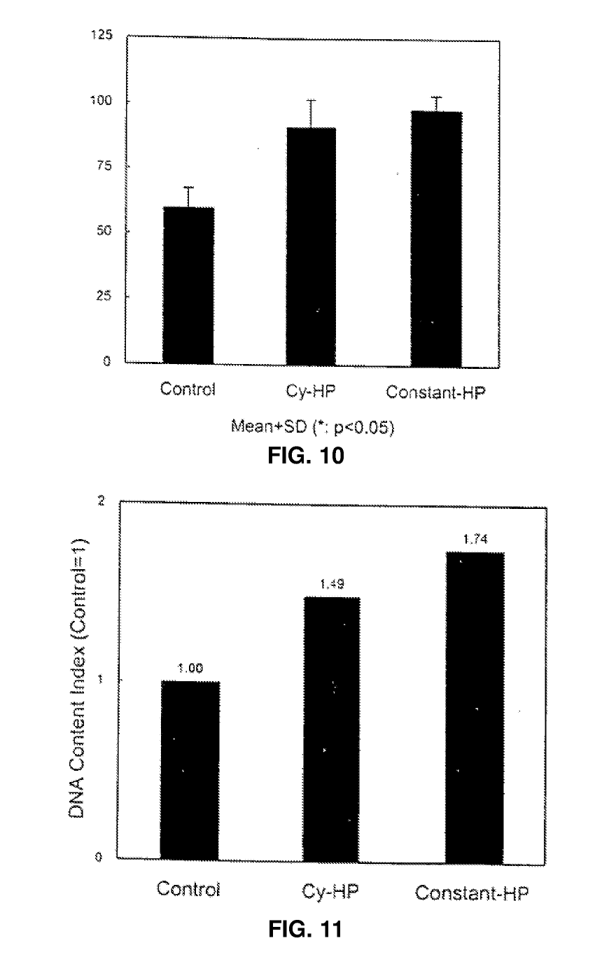

FIG. 10 shows results of glycosaminoclycan measurement.

FIG. 11 gives results of a DNA assay.

FIG. 12 shows S-GAG content.

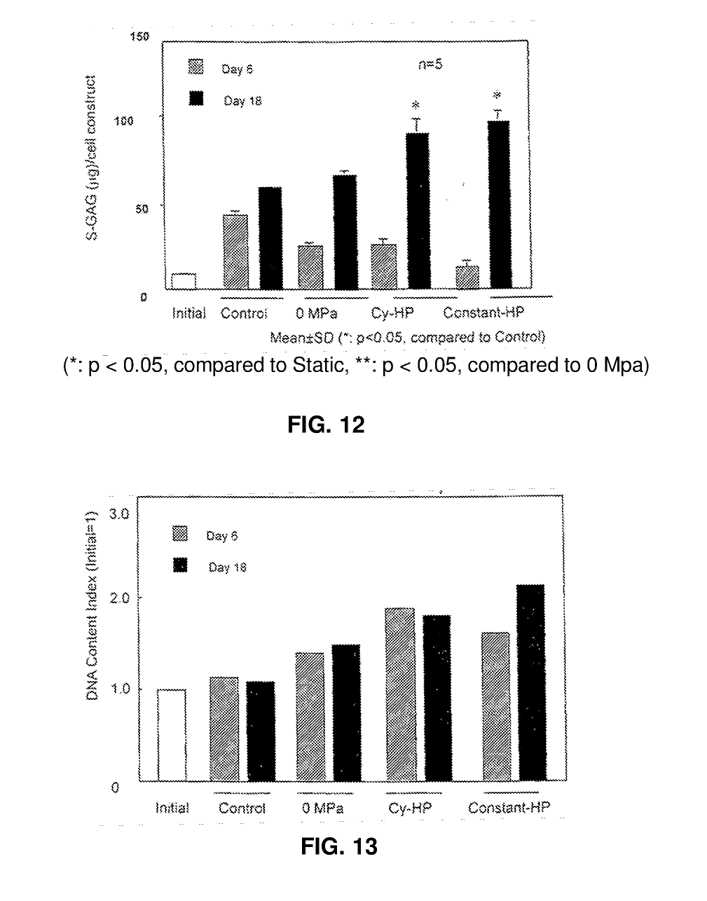

FIG. 13 shows DNA content.

All cultures were incubated at 37° C., 5% CO₂ and 20% O₂. In TESS culture, the medium flow rate was 50 μl/min. Two cell matrices from each group were harvested for histological analysis.

The matrices subjected to conditions listed in the control group, cyclic hydrostatic pressure (Cy-HP) and constant hydrostatic pressure (const-HP) groups resulted in production of 59.85, 91.05 and 97 μg/cell construct of S-GAG and 1, 1.49 and 1.74 (control=1) of DNA content Index, respectively. These results clearly show that neocartilage cultured under hydrostatic pressure, whether cyclic or constant, followed by static culture is more genetically and metabolically active than the neocartilage treated under static atmospheric conditions (controls). These results are graphically illustrated in FIGS. 10 & 11 which shows effect of hydrostatic pressure on production of sulfated glycosaminoglycan (FIG. 10) and DNA content index (FIG. 11).

FIG. 10 shows the sulfated glycosaminoglycan production in μg/cell construct wherein control represents seeded matrices subjected to atmospheric pressure, Cy-HP represents seeded matrices subjected to cyclic hydrostatic pressure (0.5 MPa) and constant-HP represent matrices subjected to constant hydrostatic pressure (0.5 MPa). There was significant increase in S-GAG production for both the cyclic (Cy-HP) and constant hydrostatic pressure (constant-HP) groups compared to atmospheric pressure (control) group. Specifically, the production of S-GAG in the control group was 59.85 μg/cell construct. In the group Cy-HP the production was 91.05 μg/cell construct. In the group constant-HP cell construct production was 97.854 μg/cell construct resulting in increase of S-GAG production to 152% for group Cy-HP and to 162% for the group constant-HP compared to the control group.

FIG. 11 shows increased production of DNA in constructs processed under cyclic or constant hydrostatic pressure.

FIG. 12 is a graph comparing effect of constant atmospheric pressure (Control) and zero MPa hydrostatic pressure (0 MPa) serving as pressure controls, 0.5 MPa cyclic hydrostatic pressure (Cy-HP) and 0.5 MPa constant hydrostatic pressure (constant-HP) at day 6 and 18 on support matrices subjected to processing in the TESS processor. All matrices were incubated at 37° C. for 18 days. The Cy-HP and constant-HP were applied for the first 6 days followed by 12 days of incubation at atmospheric pressure.

Results seen in FIG. 12 show that combination of Cy-HP or constant-HP with resting period of atmospheric pressure incubation resulted in significant (p<0.05) increase of S-GAG production in the processed matrices compared to S-GAG production observed in matrices processed at atmospheric pressure with perfusion only.

FIG. 13 shows the index of DNA content (Initial=1) in matrices subjected to static (Control), zero hydrostatic (0 MPa), cyclic (Cy-HP) or constant (Constant-HP) hydrostatic pressure for 6 day and 12 days of atmospheric pressure culture. Increase in DNA content in matrices subjected to the algorithm conditions is clearly shown in both cyclic and constant hydrostatic pressure groups. Comparison of the initial and control DNA level to DNA levels in all three groups subjected to hydrostatic pressure reveals that the DNA level in constructs subjected to the cyclic hydrostatic pressure is higher at day 6 than at day 18 and the DNA level in constructs subjected to constant hydrostatic pressure is lower at day 6 than at day 18. Highest levels of DNA is observed in matrices submitted to constant hydrostatic pressure at day 18.

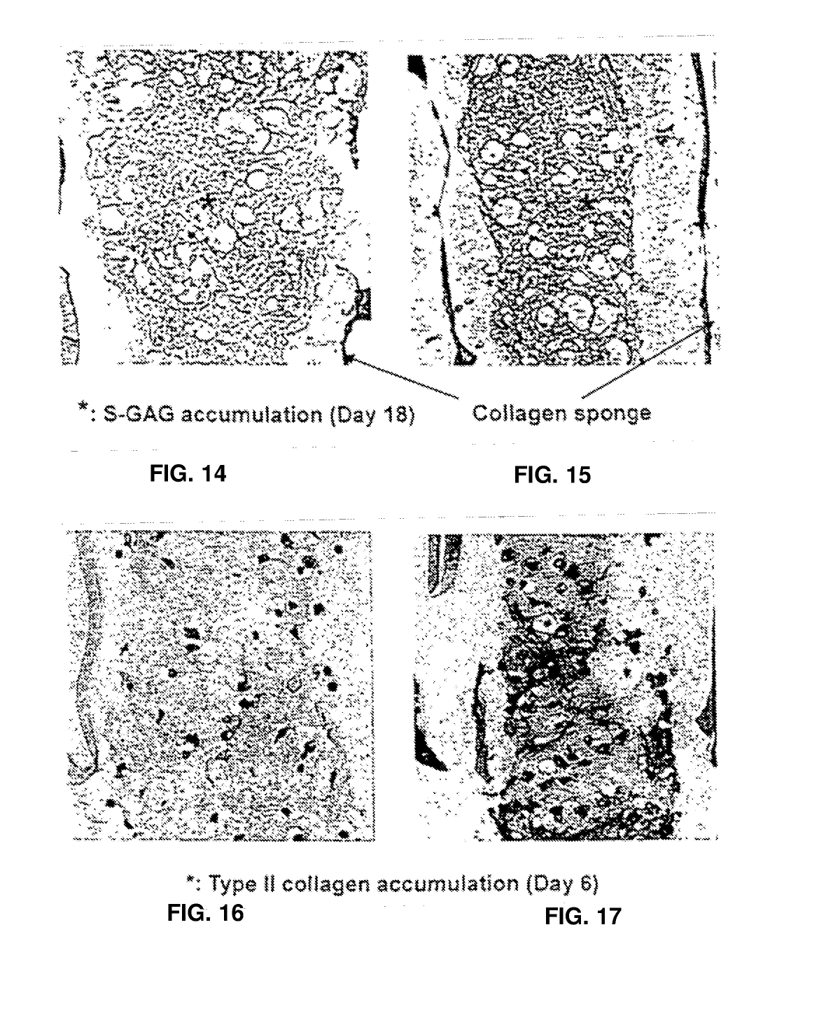

FIGS. 14 and 15 show histological evaluation of matrices by Safranin-O.

FIG. 14 shows accumulation of S-GAG on day 18 in matrices subjected to atmospheric pressure. FIG. 15 shows accumulation of S-GAG in matrices subjected to 6 days of cyclic hydrostatic pressure (Cy-HP), followed by 12 days of atmospheric pressure. The greater S-GAG accumulation in Cy-HP culture matrices is evident from the increased density of the photomicrograph clearly visible in the construct.

FIG. 16 shows accumulation of Type II collagen in matrices subjected to the atmospheric pressure.

FIG. 17 shows accumulation of Type II collagen in matrices subjected to cyclic hydrostatic pressure. Larger accumulation of Type II collagen in FIG. 17 is clearly seen.

These results demonstrate that chondrocytes may be placed in culture to coalesce into a neocartilage construct with accumulated extracellular matrix macro molecules, such as sulfated glycosaminoglycan (S-GAG).

ii. Evaluation of Effect of Perfusion Flow

The second type of study was performed in order to determine the effect of perfusion flow rate on chondrocyte proliferation (DNA content) and production of extracellular matrix (S-GAG accumulation). Results are seen in FIGS. 18 and 19.

FIG. 18 describes results of studies of the effect of the perfusion flow rate on cell proliferation measured by levels of DNA content index at day 0, 6 and 18.

FIG. 19 describes results of studies of the effect of the perfusion flow rate on cell proliferation measured by S-GAG accumulation at day 0, 6 and 18.

FIG. 18 shows that the lower perfusion rate (5 μl/min) results in higher DNA content index used as a measure for determination of cell proliferation. Specifically, the DNA content index compared to the initial DNA content index equal to 1 increased by about 50% to about 1.5 when the culture perfusion rate was 5 μl/min. The higher perfusion rate (50 μl/min) resulted in much smaller increase in DNA content index to about 1.2. Table 3 of U.S. Pub. 2014/0193468 (incorporated by reference) shows the effect of perfusion flow rate on the S-GAG production in matrices treated as outlined above where the flow rate was either 0.05 mL/min (50 μl/min) or 0.005 mL/min (5 μl/min).

TABLE 3

| | | Culture duration | | | |
|---|---|---|---|---|---|
| | Medium | In TESS | | | |
| | Perfusion | (0.5 MPA | In Incubator | Total | GAG Production |
| Group | Flow Rate | Cylic | (Atmospheric | days in | (μg/cell construct) |
| (n = 7) | (mL/min) | Pressure | Pressure) | culture | (Mean ± SD) |
| A | 0.05 mL/min | 6 days | 12 days | 18 days | 78.75 ± 6.84 |
| B | 0.005 mL/min | 6 days | 12 days | 18 days | 107.33 ± 8.53 |

All cultures were incubated at 37° C., 5% CO2 and 20% O2. In the culture, 0.5 MPa cyclic pressure at 0.5 Hz was applied to the cell matrices. Two matrices from each group were harvested for histological analysis.

As seen in Table 3 of U.S. Pub. 2014/0193468, the lower perfusion rate (5 μl/min) resulted in approximately 1.5 higher production of S-GAG than the higher perfusion rate (50 μl/min).

These results are seen in graphical form in FIG. 19. FIG. 19 is graph showing differences between S-GAG production by seeded support matrices subjected to a medium perfusion flow rate of 5 μl/min compared to matrices subjected to a medium perfusion flow rate of 50 μl/min at days 6 and 18. As seen in FIG. 19, increase in S-GAG production up to 136% (p<0.05) in matrices subjected to a slower rate of 5 μl/min.

The results summarized in FIGS. 18 and 19 clearly show a significant increase in both the DNA content index and S-GAG production in the cell construct at a flow rate of 5 μl/min compared to the flow rate 50 μl/mL. There is no significant difference in the amount of S-GAG released into the medium between the two flow rates. It is therefore possible to use lower flow rate and avoid shear.

Determination whether the combination of the perfusion flow rate with cyclic or constant hydrostatic pressure leads to increased formation of extracellular matter was also studied. Results are seen in FIGS. 20-22.

FIG. 20 illustrates a formation of extracellular matrix after 15 days culture determined in matrices treated with perfusion (5 μl/min) only.

FIG. 21 illustrates a formation of extracellular matrix after 15 days culture determined in matrices treated cyclic hydrostatic pressure 2.8 MPa at 0.015 Hz.

FIG. 22 illustrates a formation of extracellular matrix after 15 days culture determined in matrices treated constant hydrostatic pressure 2.8 MPa at 0.015 Hz as determined by toluidine blue staining. Those figures clearly show that hydrostatic pressure and medium perfusion enhances production of extracellular matrix.

iii. Evaluation of Effect of Low Oxygen Tension

The third type of study was performed in order to determine the effect of low oxygen tension on chondrocyte proliferation (DNA content) and production of extracellular matrix (S-GAG accumulation). Results are seen in Table 4 of U.S. Pub. 2014/0193468 and FIGS. 23 and 24. All cultures were incubated at 37° C., at 5% CO2. In TESS culture, the medium flow rate was 5 μl/min. Two cell matrices from each group were harvested for histological analysis.

As seen in Table 4, the lower oxygen tension (2% O2 concentration) resulted in approximately 1.7 higher production of S-GAG than higher oxygen concentration (20%) corresponding to atmospheric O2 concentration.

FIG. 23 is a graph showing differences between S-GAG production by cell constructs subjected to 2% oxygen concentration (Cy-HP) and to cyclic hydrostatic pressure followed by static pressure compared to cell constructs subjected to 20% oxygen concentration and Cy-HP followed by static pressure. As already seen in Table 4, at 2% oxygen concentration compared to 20% concentration, the production of S-GAG rose by approximately 70%.

FIG. 24 shows the DNA content index (initial=1) in cell constructs subjected to 2% or 20% oxygen concentration and Cy-HP pressure followed by static pressure. There are no significant differences in the DNA content index between 2% oxygen concentration and 20% oxygen concentration. These results indicate that the lower oxygen tension stimulates S-GAG production in cell constructs when combined with the cyclic hydrostatic culture followed by static culture. However, the cell proliferation, expressed as DNA content index, is not affected by changes in oxygen tension.

The algorithm of the invention thus comprises at least a combination of the low perfusion flow rate from about 1 to 500 μl/minute, preferably about 5 to 50 μl/minute, most preferably about 5 μl/minute, low oxygen concentration from about 1% to about 20%, preferably about 2% to about 5%, with a certain predetermined period of cyclic or constant hydrostatic pressure from zero to about 10 MPa at about 0.01 to about 1 Hz, preferably about 0.1 to about 0.5 Hz, from about zero to about 10 MPa of cyclic or constant hydrostatic pressure, preferably about 0.05 MPa to about 3 MPa at about 0.1 to about 0.5 Hz, followed by the period of a static atmospheric pressure. The algorithm conditions are applied from about 1 hour to about 90 days wherein the time for applying the hydrostatic pressure is from zero to about 24 hours per day for from about one day to about ninety days, wherein said hydrostatic pressure is preceded or followed by a period of zero to about 24 hours of a static atmospheric pressure for from about one day to about ninety days with preferred time for applying the hydrostatic cyclic or constant pressure of about 7 to 28 days followed or preceded by a period of zero to about 28 days of the atmospheric pressure.

### II. Neocartilage Composition Construct

The neocartilage composition construct is a multilayered three-dimensional structure that includes living universal chondrocytes incorporated into a cellular support matrix. The support matrix is embedded with living chondrocytes. The construct is made in vitro and ex vivo prior to implanting into the cartilage lesion. The construct is made using the method and conditions, cumulatively called the algorithm, described above, with all conditions being variable within the given ranges and depending on the intended use or on the method of delivery.

In one embodiment, the autologous or heterologous chondrocytes are cultured as described, embedded into the support matrix and processed into the neocartilage construct using predetermined medium perfusion flow rate, cyclic or constant hydrostatic pressure and reduced or increased concentration of oxygen and/or carbon dioxide. The neocartilage construct is delivered into the cartilage lesion cavity and deposited between two layers of sealant and left in situ to be integrated into the native cartilage.

### III. Method for Formation of Superficial Cartilage Layer

When the neocartilage, a neocartilage construct, or seeded support matrix produced according to procedures and conditions described above is implanted into a cartilage lesion cavity and covered with a biocompatible adhesive sealant, the resulting combination leads to a formation of a superficial cartilage layer completely overgrowing said lesion. The method is based on producing a neocartilage and neocartilage construct comprising support matrix seeded with universal chondrocytes processed according to the algorithm of the invention. Chondrocytes are typically suspended in a collagen sol which is thermo-reversible and easily changes from sol to gel at the body temperature thereby permitting external preparation of and delivery of the neocartilage construct into the lesion in form of the sol which changes its state into gel upon delivery to the lesion and warming to the body temperature.

The neocartilage construct is implanted into the lesion and covered by a layer of a biologically acceptable adhesive sealant. Optionally, the first layer of the sealant is introduced into the lesion and deposited at the bottom of the lesion. This first sealant's function is to prevent entry and to block the migration of sub-chondral and synovial cells of the extraneous components, such as blood-borne agents, cell and cell debris, etc. into the cavity and their interference with the integration of the neocartilage therein. The second sealant layer is placed over the surface of the construct. The presence of both these sealants in combination with the neocartilage construct results in successful integration of the neocartilage into the joint cartilage.

The method may be practiced in several modes and each mode involves generic steps outlined below in variable combinations.

FIG. 25 depicts a composition for cartilage repair. The composition includes a bulk implant material 1501 comprising a porous primary scaffold 1505 comprising collagen and a plurality of pores 1509. The bulk implant material 1501 further includes a secondary scaffold 1513 comprising a second collagen disposed within the plurality of pores 1509 and a plurality of living cells 1519 from a universal cell line disposed within the bulk implant material 1501. The bulk implant material is configured such that at least a first cartilage repair implant 1525, a second cartilage repair implant 1525, and a third cartilage repair implant 1527 for a plurality of different human patients may be excised from the bulk implant material. Preferably the bulk implant material is configured such that each of the plurality of different cartilage repair implants may be at least as large as a disc with a diameter of 5 mm and a thickness of 2 mm. The blacked dashed lines show where the bulk implant material 1501 may be cut to excise implants 1525, 1526—those lines likely do not actually appear on the bulk implant material 1501.

In some embodiments, the living cells 1519 are chondrocytes differentiated from pluripotent stem cells.

In a preferred embodiment, the bulk implant material 1501 comprises a sheet less than 5 mm thick and greater than a few cm by a few cm in area. The porous primary scaffold 1505 may have a substantially homogeneous defined porosity and each of the plurality of pores 1509 may have a diameter of about 300±100 μm at an upper surface 1507 and a lower surface 1515 of the sheet. The secondary scaffold 1513 should have a basic pH and include a surfactant.

The sheet may include a plurality of nanoparticles such as nutrients, growth factors, antibodies, drugs, steroids, and anti-inflammatories.

Preferably, the sheet is prepared using the plurality of living cells 1519 in a monolayer, 2D culture in the presence of a bioactive agent (e.g., TGF-β1) under conditions sufficient for inducing proliferation and differentiation of the pluripotent stem cells into the chondrocytes.

In some embodiments, the porous primary scaffold does not include any cells. In certain embodiments, the collagen and the second collagen each comprise Type I collagen.

The secondary scaffold 1513 may include a bone inducing agent such as a fibroblast growth factor (FGF), a bone morphogenic protein (BMP), insulin growth factor (IGF), and transforming growth factor beta (TGF-B).

The composition 1501 may include a fibroblast growth factor (FGF), a bone morphogenic protein (BMP), insulin growth factor (IGF), and transforming growth factor beta (TGF-B).

The plurality of living cells 1519 may include both pluripotent stem cells and universal chondrocytes differentiated from the pluripotent stem cells. For example, the plurality of living cells 1519 may include pluripotent stem cells actively differentiating into chondrocytes.

FIG. 26 diagrams a method of making implants for cartilage repair. The method includes introducing a composition comprising collagen and a plurality of living universal chondrocytes into a tissue reactor. The composition is incubated to form a bulk implant material. Preferably, the bulk implant material 1501 has a porous primary scaffold 1505 comprising collagen and a plurality of pores 1509. The bulk implant material 1501 preferably also includes a secondary scaffold 1513 comprising a second collagen disposed within the plurality of pores 1509 and a plurality of living cells 1519 from a universal cell line disposed within the bulk implant material 1501

A first implant 1525 is excised from the bulk implant material. The first implant 1525 includes a first portion of the living universal chondrocytes and is suitable for implantation into a first human patient.

A second implant 1526 is also excised from the bulk implant material. The second implant comprises a second portion of the living universal chondrocytes and is suitable for implantation into a second human patient.

General way to practice the method for repair and restoration of damaged, injured, diseased or aged cartilage is described below.

### A. Preparing Neocartilage, Neocartilage Construct or Chondrocyte Support Matrix

The following section describes methods for implanting of neocartilage constructs prepared with any of the above methods, including with or with growth factors and with or without hydrostatic pressure.

B. Depositing the First and Second Sealant into the Lesion

This step involves introducing a first and a second layer of a first and a second biologically acceptable sealant into a cartilage lesion. The first and second sealants may be the same or different. It is to be understood that the utilization of the first bottom layer is optional and that the method for a formation of the superficial cartilage layer is enabled without the first layer.

Specifically, this step involves deposition of the first sealant at the bottom of the lesion and of the second sealant over the lesion. The first and the second sealants can be the same or different, however, both the first and the second sealants must have certain definite properties to fulfill their functions.

The first sealant, deposited into the cavity before the neocartilage is deposited, acts as a protector of the lesion cavity integrity, that is, it protects the lesion cavity not only from extraneous substances but it also protect this cavity from formation of the fibrocartilage in the interim when the cavity is filled with a space-holding gel in expectation of implantation of the neocartilage after processing. The second sealant acts as a protector of the lesion cavity on the outside as well as a protector of the neocartilage construct deposited within a cavity formed between the two sealants and as well as an initiator of the formation of the superficial cartilage layer.

i. First Sealant

The optionally deposited first sealant forms an interface between the introduced neocartilage construct and the native cartilage. The first sealant, deposited at the bottom of the lesion, must be able to protect the construct from and prevent chondrocyte migration into the sub-chondral space. Additionally, the first sealant prevents the infiltration of blood vessels and undesirable cells and cell debris into the neocartilage construct and it also prevents formation of the fibrocartilage.

ii. Second Sealant

The second sealant acts as a protector of the neocartilage construct or the lesion cavity on the outside and is typically deposited over the lesion either before or after the neocartilage is deposited therein and in this way protects the integrity of the lesion cavity from any undesirable effects of the outside environment, such as invading cells or degradative agents and seals the space holding gel in place before the neocartilage is deposited therein. The second sealant also acts as a protector of the neocartilage construct implanted within a cavity formed between the two sealants. In this way, the second sealant may be deposited after the neocartilage is implanted over the first sealant and seal the neocartilage within the cavity or it may be deposited over the space holding gel. The third function of the second sealant is as an initiator or substrate for the formation of a superficial cartilage layer. Studies performed during the development of this invention discovered that when the second sealant was deposited over the cartilage lesion, a growth of the superficial cartilage layer occurred as an extension of the native superficial cartilage layer. This superficial cartilage layer is particularly well-developed when the lesion cavity is filled with the space-holding or thermo-reversible gel thereby leading to the conclusion that such a gel might provide a substrate for the formation of such superficial cartilage layer.

iii. First and Second Sealant Properties

The first or second sealant of the invention must possess the following characteristics:

Sealant must be biologically acceptable, easy to use and possess required adhesive and cohesive properties. The sealant is biologically compatible with tissue, be non-toxic, not swell excessively, not be extremely rigid or hard, as this could cause abrasion of or extrusion of the sealant from the tissue site, must not interfere with the formation of new cartilage, or promote the formation of other interfering or undesired tissue, such as bone or blood vessels and must resorb and degrade by an acceptable pathway or be incorporated into the tissue.

The sealant must rapidly gel from a flowable liquid or paste to a load-bearing gel within 3 to 15 minutes, preferably within 3-5 min. Longer gelation times are not compatible with surgical time constraints. Additionally, the overall mode of use should be relatively simple because complex procedures will not be accepted by surgeons.

Adhesive bonding is required to attach the sealant formulation to tissue and to seal and support such tissue. Minimal possessing peel strengths of the sealant should be at least 3 N/m and preferably 10 to 30 N/m. Additionally, the sealant must itself be sufficiently strong so that it does not break or tear internally, i.e., it must possess sufficient cohesive strength, measured as tensile strength in the range of 0.2 MPa, but preferably 0.8 to 1.0 MPa. Alternatively, a lap shear measurement may be given to define the bond strength of the formulation should have values of at least 0.5 N/cm² and preferably 1 to 6 N/cm².

Sealants possessing the required characteristics are typically polymeric. In the un-cured, or liquid state, such sealant materials consist of freely flowable polymer chains which are not cross-linked together, but are neat liquids or are dissolved in physiologically compatible aqueous buffers. The polymeric chains also possess side chains or available groups which can, upon the appropriate triggering step, react with each other to couple, or cross-link the polymer chains together. If the polymer chains are branched, i.e., comprising three or more arms on at least one partner, the coupling reaction leads to the formation of a network which is infinite in molecular weight, i.e., a gel.

The formed gel has cohesive strength dependent on the number of inter-chain linkages, the length (molecular weight) of the chains between links, the degree of inclusion of solvent in the gel, the presence of reinforcing agents, and other factors. Typically, networks in which the molecular weight of chain segments between junction points (cross-link bonds) is 100-500 Daltons are tough, strong, and do not swell appreciably. Networks in which the chain segments are 500-2500 Daltons swell dramatically in aqueous solvents and become mechanically weak. In some cases the latter gels can be strengthened by specific reinforcer molecules; for example, the methylated collagen reinforces the gels formed from 4-armed PEGs of 10,000 Daltons (2500 Daltons per chain segment).

The gel's adhesive strength permits bonding to adjacent biological tissue by one or more mechanisms, including electrostatic, hydrophobic, or covalent bonding. Adhesion can also occur through mechanical inter-lock, in which the uncured liquid flows into tissue irregularities and fissures, then, upon solidification, the gel is mechanically attached to the tissue surface. At the time of use, some type of triggering action is required. For example, it can be the mixing of two reactive partners, it can be the addition of a reagent to raise the pH, or it can be the application of heat or light energy.

Once the sealant is in place, it must be non-toxic to adjacent tissue, and it must be incorporated into the tissue and retained permanently, or removed, usually by hydrolytic or enzymatic degradation. Degradation can occur internally in the polymer chains, or by degradation of chain linkages, followed by diffusion and removal of polymer fragments dissolved in physiological fluids.

Another characteristic of the sealant is the degree of swelling it undergoes in the tissue environment. Excessive swelling is undesirable, both because it creates pressure and stress locally, and because a swollen sealant gel loses tensile strength, due to the plasticizing effect of the imbibed solvent (in this case, the solvent is physiological fluid). Gel swelling is modulated by the hydrophobicity of the polymer chains. In some cases it may be desirable to derivatize the base polymer of the sealant so that it is less hydrophilic. For example, one function of methylated collagen containing sealant is presumably to control swelling of the gel. In another example, the sealant made from penta-erythritol tetra-thiol and polyethylene glycol diacrylate can be modified to include polypropylene glycol diacrylate, which is less hydrophilic than polyethylene glycol. In a third example, sealants containing gelatin and starch can also be methylated both on the gelatin and on the starch, again to decrease hydrophilicity.

iv. Suitable Sealants

Sealants suitable for purposes of this invention include the sealants prepared from gelatin and di-aldehyde starch triggered by mixing aqueous solutions of gelatin and dialdehyde starch which spontaneously react and gel. The gel bonds to tissue through a reaction of aldehyde groups on starch molecules and amino groups on proteins of tissue, with an adhesive bond strength to up to 100 N/m and an elastic modulus of 8×10⁶ Pa, which is a characteristic of a relatively tough, strong material. After swelling in physiological fluids this cohesive strength declines. The gelled sealant is degraded by enzymes that cleave the peptide bonds of gelatin and the glycosidic bonds of starch.

Another acceptable sealant is made from a copolymer of polyethylene glycol and poly-lactide or -glycolide, further containing acrylate side chains and gelled by light, in the presence of some activating molecules. The linkage is formed by free-radical chemistry. The gel bonds to tissue by mechanical interlock, having flowed into tissue surface irregularities prior to curing. The sealant degrades from the tissue by hydrolytic cleavage of the linkage between polyethylene glycol chains, which then dissolve in physiological fluids and are excreted.

The acceptable sealant made from periodate-oxidized gelatin remains liquid at acid pH, because free aldehyde and amino groups on the gelatin cannot react. To trigger gelation, the oxidized gelatin is mixed with a buffer that raises the pH, and the solution gels. Bonding to tissue is through aldehyde groups on the gelatin reacting with amino groups on tissue. After gelation, the sealant can be degraded enzymatically, due to cleavage of peptide bonds in gelatin.

Still another sealant made from a 4-armed pentaerythritol thiol and a polyethylene glycol diacrylate is formed when these two neat liquids (not dissolved in aqueous buffers) are mixed. The rate of gelation is controlled by the amount of a catalyst, which can be a quaternary amino compound, such as tri-ethanolamine. A covalent linkage is formed between the thiol and acrylate, to form a thio-ether bond. The final gel is firm and swells very little. The tensile strength of this gel is high, about 2 MPa, which is comparable to that of cyanoacrylate acceptable Superglue. Degradation of such gels in vivo is slow. Therefore, the gel may be encapsulated or incorporated into tissue.

Another example is the composition, preferred for use in this invention, that contains 4-armed tetra-succinimidyl ester or tetra-thiol derivatized PEG, plus methylated collagen. The reactive PEG reagents in powder form are mixed with the viscous, fluid methylated collagen (previously dissolved in water); this viscous solution is then mixed with a high pH buffer to trigger gelation. The tensile strength of this cured gel is about 0.3 MPa. Degradation presumably occurs through hydrolytic cleavage of ester bonds present in the succinimidyl ester PEG, releasing the soluble PEG chains which are excreted.

In general, a sealant useful for the purposes of this application has adhesive, or peel strengths at least 10 N/m and preferably 100 N/cm; it needs to have tensile strength in the range of 0.2 MPa to 3 MPa, but preferably 0.8 to 1.0 MPa. In so-called “lap shear” bonding tests, values of 0.5 up to 4-6 N/cm² are characteristic of strong biological adhesives.

Such properties can be achieved by a variety of materials, both natural and synthetic. Six examples include: (1) gelatin and di-aldehyde starch (International Patent Publication Number WO 97/29715; 21 Aug. 1997); (2) 4-armed penta-erythritol tetra-thiol and polyethylene glycol diacrylate (U.S. Pat. No. 7,744,912); (3) photo-polymerizable polyethylene glycol-co-poly(a-hydroxy acid) diacrylate macromers (U.S. Pat. No. 5,410,016); (4) periodate-oxidized gelatin (U.S. Pat. No. 5,618,551); (5) serum albumin and di-functional polyethylene glycol derivatized with maleimidyl, succinimidyl, phthalimidyl and related active groups (U.S. Pat. No. 5,583,114) and (6) 4-armed polyethylene glycols derivatized with succinimidyl ester and thiol, plus methylated collagen, referred to as “CT3” (U.S. Pat. No. 6,312,725).

Various other sealant formulations are available commercially or are described in the literature. Some may not be suitable for practicing this invention for a variety of reasons. For example, fibrin sealant is unsuitable because it interferes with the formation of cartilage. Cyanoacrylate, or Superglue, is extremely strong but it might exhibit toxic reactions in tissue.

Un-reinforced hydrogels of various types typically exhibit tensile strengths of lower than 0.02 MPa, which is too weak to support the adhesion required for the purpose of this application because such gels will swell too much, tear too easily, and break down too rapidly.

It is worth noting that it is not the presence or absence of particular protein or polymer chains, such as gelatin or polyethylene glycol, which necessarily govern the mechanical strength and degradation pattern of the sealant. The mechanical strength and degradation pattern are controlled by the cross-link density of the final cured gel, by the types of degradable linkages which are present, and by the types of modifications and the presence of reinforcing molecules, which may affect swelling or internal gel bonding.

v. Preferred Sealants

The first or second sealant of the invention must be a biologically acceptable, typically rapidly gelling synthetic compound having adhesive, bonding and/or gluing properties, and is typically a hydrogel, such as derivatized polyethylene glycol (PEG) which is preferably cross-linked with a collagen compound, typically alkylated collagen. Sealant should have a tensile strength of at least 0.3 MPa. Examples of suitable sealants are tetra-hydrosuccinimidyl or tetra-thiol derivatized PEG, or a suitable PEG hydrogel sealant such as the PEG hydrogel sealant sold under the trademark DURASEAL by Covidien (Waltham, Mass.) or the sealant sold under the trademark COSEAL by Baxter International, Inc. (Deerfield, Ill.); see also Wallace et al., 2001, A tissue sealant based on reactive multifunctional polyethylene glycol, J. Biomed. Mater. Res (Appl. Biomater.) 58:545-555. Other suitable compounds include the rapid gelling biocompatible polymer compositions described in U.S. Pat. No. 6,312,725, incorporated by reference. Additionally, the sealant may be two or more-part polymers compositions that rapidly form a matrix where at least one of the compounds is polymer, such as, polyamino acid, polysaccharide, polyalkylene oxide or polyethylene glycol and two parts are linked through a covalent bond and cross-linked PEG with methyl collagen, commercially available.

The sealant of the invention typically gels rapidly upon contact with tissue, particularly with tissue containing collagen. The second sealant may or may not be the same as the first sealant. Both the first and the second is preferably a cross-linked polyethylene glycol hydrogel with methyl-collagen, which has adhesive properties.

### C. Implanting the Neocartilage Construct

Next step in the method of the invention comprises implanting said neocartilage into a lesion cavity formed under the second sealant or between two layers of sealants, said cavity either filled with neocartilage construct deposited therein or, optionally, with a space holding thermo-reversible gel (SHTG) deposited into said cavity as a sol at temperatures between about 5 to about 30° C. wherein, within said cavity and at the body temperature, said SHTG converts the sol into gel and in this form the SHTG holds the space for introduction of the neocartilage construct and provides protection for the neocartilage and wherein its presence further promotes in situ formation of de novo superficial cartilage layer covering the cartilage lesion.

The above step is versatile in that the neocartilage may be deposited into a lesion cavity after the first sealant is deposited but before the second sealant is deposited over it or the first and second sealants may be deposited first and the cavity is filled with the space-holding thermo-reversible gel for the interim period when the neocartilage is cultured and processed or it may be deposited into the lesion cavity without the first sealant and covered with the second sealant.

The neocartilage is either autologous or heterologous and is prepared using any of the expansion and culturing methods described above.

D. Removing Gel from the Lesion Cavity

The neocartilage is deposited into the cavity either before or after the formation of the superficial cartilage layer. In all cases when the first sealant is used, the first sealant is deposited first. In one embodiment, the neocartilage construct containing, typically, the heterologous neocartilage might be deposited on the top of the first sealant layer and immediately covered by the second sealant layer. In such an instance, the neocartilage is left in the cavity until the superficial cartilage layer is formed and the neocartilage is integrated into the surrounding cartilage. Then, depending on the material used for neocartilage construct, the sponge gel or thermo-reversible gelling hydrogel are left in the cavity to disintegrate.

In the instance when the two sealants are deposited first, the space within the lesion cavity is optionally filled with a polymer gel, such as the space-holding thermo-reversible gel. Such gel is left in the cavity until the neocartilage construct is cultured, processed and ready to be implanted. Since such thermo-reversible gel might or might not be completely or partially degraded during this time, it may be removed from the cavity by cooling the lesion to about 50° C., at which temperature the gel becomes a sol, and by removing said sol from the cavity, for example, by injection. Using the same process of cooling the solid gel of the neocartilage, the process may be reversed for introduction of the neocartilage construct into said lesion cavity wherein, after the sol is warmed into the body temperature, the sol is converted into a solid gel.

Thus, the primary premise of this process is that the removal and/or introduction of the space holding gel or introduction of neocartilage construct proceeds at the cold temperature where the composition is in the sol state and converts into solid gel at warmer temperatures. In this way the gel may be removed from the cavity as the sol after the neocartilage integration and formation of superficial cartilage layer.

### E. Generation of the Superficial Cartilage Layer

A combination of the neocartilage construct comprising the neocartilage suspended in the thermo-reversible gel or support matrix embedded with chondrocytes with the adhesive polymeric second sealant leads to overgrowth and complete or almost complete sealing of the lesion cavity. Alternatively, depending on the surface chemistry of the thermo-reversible gel, the superficial layer could grow directly over the neocartilage construct if such surface chemistry is propitious to such growth.

Typically, a biologically acceptable second sealant, preferably a cross-linked PEG hydrogel with methyl collagen sealant, is deposited either over the neocartilage construct implanted into the lesion cavity or is deposited over the lesion before the neocartilage construct is deposited therein. The second sealant acts as an initiator for formation of the superficial cartilage layer which in time completely overgrows the lesion. The superficial cartilage layer in several weeks or months completely covers the lesion and permits integration of the neocartilage of the neocartilage construct or chondrocytes embedded within the support matrix into the native surrounding cartilage substantially without formation of fibrocartilage.

Formation of the superficial cartilage layer is a very important aspect of the healing of the cartilage and its repair and regeneration.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of making implants for cartilage repair, the method comprising:
introducing a composition comprising collagen and a plurality of living universal chondrocytes into a tissue reactor;
incubating the composition to form a bulk implant material;
excising a first implant from the bulk implant material, wherein the first implant comprises a first portion of the living universal chondrocytes and is suitable for implantation into a first human patient; and
excising a second implant from the bulk implant material, wherein the second implant comprises a second portion of the living universal chondrocytes and is suitable for implantation into a second human patient.

2. The method of claim 1, further comprising differentiating pluripotent stem cells into the living universal chondrocytes prior to the introducing step.

3. The method of claim 1, wherein the plurality of living universal chondrocytes are differentiated pluripotent stem cells.

4. The method of claim 3, wherein the composition further comprises a porous primary scaffold comprising the collagen and a plurality of pores, and the introducing step further comprises introducing a solution comprising a second collagen and the plurality of living universal chondrocytes into the plurality of pores.

5. The method of claim 4, wherein incubating the composition stabilizes the solution to form a fibrous, cross-linked network comprising the second collagen within the plurality of pores.

6. The method of claim 5, wherein the bulk implant material comprises a sheet less than 5 mm thick and greater than a few cm by a few cm in area.

7. The method of claim 6, further comprising harvesting at least four different implants for at least four different human patients from the sheet.

8. The method of claim 6, wherein the sheet is prepared using the universal cells in a monolayer, 2D culture in the presence of a bioactive agent under conditions sufficient for inducing proliferation and differentiation of the pluripotent stem cells into the universal chondrocytes.

9. The method of claim 7, wherein the collagen and the second collagen each comprise Type I collagen.

10. The method of claim 9, wherein the porous primary scaffold has a substantially homogeneous defined porosity and wherein each of the plurality of pores have a diameter of about 300±100 μm at an upper surface and a lower surface of the sheet, and wherein the solution further includes a chondrogenic growth factor.

11. A composition for cartilage repair, the composition comprising:
a bulk implant material comprising
a porous primary scaffold comprising collagen and a plurality of pores,
a secondary scaffold comprising a second collagen disposed within the plurality of pores, and
a plurality of living cells from a universal cell line disposed within the bulk implant material,
wherein the bulk implant material is configured such that a plurality of different cartilage repair implants for a plurality of different human patients may be excised from the bulk implant material.

12. The composition of claim 11, wherein the bulk implant material is configured such that each of the plurality of different cartilage repair implants may be at least as large as a disc with a diameter of 5 mm and a thickness of 2 mm.

13. The composition of claim 12, wherein the plurality of living cells include chondrocytes differentiated from pluripotent stem cells.

14. The composition of claim 13, wherein the bulk implant material comprises a sheet less than 5 mm thick and greater than a few cm by a few cm in area.

15. The composition of claim 14, wherein the porous primary scaffold has a substantially homogeneous defined porosity and wherein each of the plurality of pores have a diameter of about 300±100 μm at an upper surface and a lower surface of the sheet

16. The composition of claim 15, wherein the sheet is prepared using the plurality of living cells in a monolayer, 2D culture in the presence of a bioactive agent under conditions sufficient for inducing proliferation and differentiation of the pluripotent stem cells into the chondrocytes.

17. The composition of claim 14, wherein the collagen and the second collagen each comprise Type I collagen.

18. The composition of claim 14, further comprising a transforming growth factor beta 1.

19. The composition of claim 14, wherein the plurality of living cells comprises pluripotent stem cells and chondrocytes differentiated from pluripotent stem cells.

20. The composition of claim 14, wherein the plurality of living cells comprises pluripotent stem cells actively differentiating into chondrocytes.
